(19) United States
(12) Patent Application Publication
(10) Pub. No.: US 2011/0030099 A1
(43) Pub. Date: Feb. 3, 2011

(54) PLANTS AND MODULATORS FOR IMPROVED DROUGHT TOLERANCE

(76) Inventors: John Andrew Ryals, Durham, NC (US); Lining Guo, Durham, NC (US); Kay Ann Lawton, Durham, NC (US); Xi Chen, Durham, NC (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(21) Appl. No.: 12/837,905

(22) Filed: Jul. 16, 2010

Related U.S. Application Data
(60) Provisional application No. 61/226,517, filed on Jul. 17, 2009.

(51) Int. Cl. A01H 1/06; A01H 5/00

(52) U.S. Cl. 800/285; 800/278; 800/298

(57) ABSTRACT

Methods of conferring increased drought tolerance to a plant by reducing or inhibiting ammonia accumulation, and plants produced thereby.

### RELATED APPLICATIONS

The following application claims priority to U.S. Provisional Application No. 61/226,517; filed Jul. 17, 2009 whose contents in its entirety is herein incorporated by reference.

### FIELD OF THE INVENTION

The present invention generally relates to methods and compositions for improved drought tolerance in plants. More particularly, the methods and compositions pertain to prevention or inhibition of ammonia accumulation during plant responses to drought to thereby confer improved drought tolerance.

### BACKGROUND OF THE INVENTION

One of the main objects of plant cultivation is generation of plants with improved drought tolerance. Drought tolerance is a complex trait that involves the contribution of many genes and their interactions, which are also affected by growth conditions. Understanding gene networks associated with biochemical and physiological responses to drought is an important step for germplasm improvement.

The instant application describes features of drought tolerance related to ammonia toxicity and methods of enhancing drought tolerance by decreasing ammonia accumulation under drought conditions. In particular, the instant application provides methods of improving drought tolerance in plants by limiting ammonia accumulation. For example, drought tolerance may be achieved by overexpressing in a plant nucleic acids and proteins encoding enzymes involved in glutamine and asparagine synthesis, or by otherwise activating these enzymes. As an additional example, drought tolerance may be conferred to a plant by expressing in the plant inhibitory nucleic acids that result in reduced expression of enzymes involved in allantoin catabolism, or by otherwise inhibiting such enzymes. Also provided by the instant application are methods of making drought tolerant plants and plants and hybrids produced thereby.

### SUMMARY OF THE INVENTION

The present invention provides methods of improving or increasing drought tolerance in a plant comprising reducing levels of or inhibiting accumulation of ammonia in the plant. For example, the reduction in ammonia is characterized by increased levels of glutamine, asparagine, and/or allantoin. This may be accomplished by increasing expression or activity levels of one or more of glutamine synthetase, glutamate synthetase, aspartate synthetase, and asparagine synthetase. Reducing or inhibiting accumulation of ammonia can also be accomplished by decreasing expression or activity levels of one or more of allantoinase, allantoate amidohydrolase, and ureidoglycolate amidohydrolase, for example, by expressing inhibitory nucleic acids or by disrupting a genomic locus encoding allantoinase, allantoate amidohydrolase, or ureidoglycolate amidohydrolase. Still further, methods of improving drought tolerance in a plant can comprise contacting the plant with an agent that activates or enhances the activity of one or more of glutamine synthetase, glutamate synthetase, aspartate synthetase, and asparagine synthetase, and/or contacting the plant with an agent that inhibits or reduces the activity of one or more of allantoinase, allantoate amidohydrolase, and ureidoglycolate amidohydrolase.

The present invention further provides methods of identifying agents for improving drought tolerance in a plant. As one example, the method can comprise the steps of: (a) providing a cell expressing glutamine synthetase, glutamate dehydrogenase, aspartate aminotransferase, or asparagine synthetase; (b) contacting the cell with one or more test agents or a control agent; (c) assaying expression of a glutamine synthetase, glutamate dehydrogenase, aspartate aminotransferase, or asparagine synthetase nucleic acid or protein; and (d) selecting a test agent that induces elevated expression of the glutamine synthetase, glutamate dehydrogenase, aspartate aminotransferase, or asparagine synthetase nucleic acid or protein when contacted the cell is contacted with the test agent as compared to the control agent; whereby an agent for improving or increasing drought tolerance in a plant is identified. As another example, the method can comprise the steps of: (a) providing a cell expressing glutamine synthetase, glutamate dehydrogenase, aspartate aminotransferase, or asparagine synthetase; (b) contacting the cell with one or more test agents or a control agent; (c) assaying a level of glutamine or asparagine; and (d) selecting a test agent that induces an elevated level of glutamine or asparagine when the cell is contacted with the test agent as compared to the control agent; whereby an agent for improving or increasing drought tolerance in a plant is identified. As another example, the method can comprise the steps of: (a) providing a cell expressing allantoinase, allantoate amidohydrolase, or ureidoglycolate amidohydrolase; (b) contacting the cell with one or more test agents or a control agent; (c) assaying expression of an allantoinase, allantoate amidohydrolase, or ureidoglycolate amidohydrolase nucleic acid or protein; and (d) selecting a test agent that induces decreased expression of the allantoinase, allantoate amidohydrolase, or ureidoglycolate amidohydrolase nucleic acid or protein when contacted the cell is contacted with the test agent as compared to the control agent; whereby an agent for improving or increasing drought tolerance in a plant is identified. As another example, the method can comprise the steps of: (a) providing a cell expressing allantoinase, allantoate amidohydrolase, or ureidoglycolate amidohydrolase; (b) contacting the cell with one or more test agents or a control agent; (c) assaying a level of allantoin; and (d) selecting a test agent that induces an increased level of allantoin when the cell is contacted with the test agent as compared to the control agent; whereby an agent for improving or increasing drought tolerance in a plant is identified.

In one embodiment allantoin is accumulated in plant vegetative material by suppressing the endogenous expression of either one or more of the plant endogenous enzymes selected of the group comprising allantoinase, allantoate amidohydrolase, or ureidoglycolate amidohydrolase. As used herein, the term “allantoin catabolic enzymes” or “allantoin degradation enzymes” interchangeably refer to any enzyme that degrades the molecule allantoin and/or its subsequent byproducts in plants (e.g. allantoinase, allantoate amidohydrolase, ureidoglycolate amidohydrolase and etc). In one aspect allantoinase, allantoate amidohydrolase, or ureidoglycolate amidohydrolase RNAi's are operably linked to a drought inducible regulatory element which will only drive the transcription of the respective allantoin catabolic enzyme during times of drought, drought induced elevated levels of ammonium or in the presence of abscisic acid (ABA). In some aspects by suppressing allantoin catabolic enzymes only during times of drought may provide a means to avoid negative agronomic effects. Herein, “negative agronomic effects” refer to any phenotypic or genotypic property that in some way negatively affects the transgenic plant's health, yield, commercial value, composition or reproductive capabilities (i.e. seed generation and/or seed germination rates). For example, maize plants having a dwarf phenotype would be considered to have a negative agronomic effect. In addition, a plant having for example a combination of or any one of reduced seed yield, reduced biomass, reduced chlorophyll content, shriveled seed, tissue death, sterility or reduced germination rates would be considered to have a negative agronomic effect herein.

In some aspects of the invention it is understood that one may wish to downregulate an allantoin degradation enzyme in a specific plant tissue and/or organelle. For example, it is known in the art that allantoin is stored in the peroxisomes of plants (Corpas et al. (1987) Plant Physiology 151, pp. 246-250). It is also proposed that allantoin is transported to the endoplasmic reticulum for further ureide catabolism (Werner et al. (2008) Plant Physiology 146, pp. 418-430). In some preferred embodiments, it may be necessary to specifically target polynucleotides that suppress the allantoin degradation pathway to the ER and/or to plant peroxisomes to produce plants with increased drought resistance and/or tolerance or plants having increased yield. It may be beneficial in some embodiments to target polynucleotides that suppress the allantoin degradation pathway to any one of or a combination of the seed, epidermis, root, vascular tissue, meristem, cambium, cortex, pith, leaf, or flower.

### BRIEF DESCRIPTION OF THE DRAWINGS

FIGS. 1A-1D are line graphs showing the levels of asparagine (FIG. 1A), glutamine (FIG. 1B), allantoin (FIG. 1C), and proline (FIG. 1D) at each of eight pre-flowering time points as described in Example 2. Data for each of two maize varieties grown under full irrigation (CK) or drought (DT) conditions is shown. Avg-R, average raw mass spectrometry ion counts for 4 plants with R genotype; Avg-S, average raw mass spectrometry ion counts for 4 plants with S genotype. Under drought conditions, levels of all four compounds were increased.

### DETAILED DESCRIPTION

The instant application provides methods of improving drought tolerance in plants by limiting ammonia accumulation. For example, drought tolerance may be achieved by overexpressing in a plant nucleic acids and proteins encoding enzymes involved in glutamine and asparagine synthesis, or by otherwise activating these enzymes. Drought tolerance may be conferred to a plant by expressing in the plant inhibitory nucleic acids that result in reduced expression of enzymes involved in allantoin catabolism, or by otherwise inhibiting such enzymes. Also provided by the instant application are methods of making drought tolerant plants and plants and hybrids produced thereby.

It is to be understood that this invention is not limited to the particular methodology, protocols, cell lines, plant species or genera, constructs, and reagents described as such. It is also to be understood that the terminology used herein is for the purpose of describing particular embodiments only, and is not intended to limit the scope of the present invention. As used herein the singular forms “a”, “and”, and “the” include plural reference unless the context clearly dictates otherwise. Thus, for example, reference to “a vector” is a reference to one or more vectors and includes equivalents thereof known to those skilled in the art.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent.

As used herein, the word “or” means any one member of a particular list and also includes any combination of members on that list.

“Antisense inhibition” refers to the production of antisense RNA transcripts capable of suppressing the expression of protein from an endogenous gene or a transgene.

“Cis-element” refers to a cis-acting transcriptional regulatory element that confers an aspect of the overall control of gene expression. A cis-element may function to bind transcription factors, trans-acting protein factors that regulate transcription. Some cis-elements bind more than one transcription factor, and transcription factors may interact with different affinities with more than one cis-element. Cis-elements can be identified by a number of techniques, including deletion analysis, i.e., deleting one or more nucleotides from the 5′ end or internal to a promoter; DNA binding protein analysis using DNAase I foot printing, methylation interference, electrophoresis mobility-shift assays, in vivo genomic foot printing by ligation-mediated PCR, and other conventional assays; or by DNA sequence similarity analysis with known cis-element motifs by conventional DNA sequence comparison methods. The fine structure of a cis-element can be further studied by mutagenesis (or substitution) of one or more nucleotides or by other conventional methods. Cis-elements can be obtained by chemical synthesis or by isolation from promoters that include such elements, and they can be synthesized with additional flanking nucleotides that contain useful restriction enzyme sites to facilitate subsequence manipulation.

“Chimeric” is used to indicate that a DNA sequence, such as a vector or a gene, is comprised of two or more DNA sequences of distinct origin that are fused together by recombinant DNA techniques resulting in a DNA sequence, which does not occur naturally.

“Chromosomally-integrated” refers to the integration of a foreign gene or DNA construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation, or splicing, of the RNA within the cell to create the mature mRNA that can be translated into a protein.

“Constitutive promoter” refers to a promoter that is able to express the gene that it controls in all or nearly all of the plant tissues during all or nearly all developmental-stages of the plant, thereby generating “constitutive expression” of the gene.

“Co-suppression” and “sense suppression” refer to the production of sense RNA transcripts capable of suppressing the expression of identical or substantially identical transgene or endogenous genes.

“Contiguous” is used herein to mean nucleic acid sequences that are immediately preceding or following one another.

“Expression” refers to the transcription and stable accumulation of mRNA. Expression may also refer to the production of protein.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components.

The “expression pattern” of a promoter (with or without an enhancer) is the pattern of expression that shows where in the plant and in what developmental stage the promoter initiates transcription. Expression patterns of a set of promoters are said to be complementary when the expression pattern of one promoter shows little overlap with the expression pattern of the other promoter.

“Gene” refers to a nucleic acid fragment that expresses mRNA, functional RNA, or specific protein, including regulatory sequences. The term “Native gene” refers to a gene as found in nature. The term “chimeric gene” refers to any gene that contains 1) DNA sequences, including regulatory and coding sequences that are not found together in nature or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature. A “transgene” refers to a gene that has been introduced into the genome by transformation and is stably maintained. Transgenes may include, for example, genes that are either heterologous or homologous to the genes of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but one that is introduced into the organism by gene transfer.

“Gene silencing” refers to homology-dependent suppression of viral genes, transgenes, or endogenous nuclear genes. Gene silencing may be transcriptional, when the suppression is due to decreased transcription of the affected genes, or post-transcriptional, when the suppression is due to increased turnover (degradation) of RNA species homologous to the affected genes. (English, et al., 1996, Plant Cell 8:179-1881). Gene silencing includes virus-induced gene silencing (Ruiz et al., 1998, Plant Cell 10:937-946).

“Genetically stable” and “heritable” refer to chromosomally-integrated genetic elements that are stably maintained in the plant and stably inherited by progeny through successive generations.

“Heterologous DNA Sequence” is a DNA sequence not naturally associated with a host cell into which it is introduced, including non-naturally occurring multiple copies of a naturally occurring DNA sequence.

“Inducible promoter” refers to those regulated promoters that can be turned on in one or more cell types by an external stimulus, such as a chemical, light, hormone, stress, or a pathogen. The term “nucleic acid” refers to a polynucleotide of high molecular weight which can be single-stranded or double-stranded, composed of monomers (nucleotides) containing a sugar, phosphate and a base which is either a purine or pyrimidine. A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. A “genome” is the entire body of genetic material contained in each cell of an organism. The term “nucleotide sequence” refers to a polymer of DNA or RNA which can be single- or double-stranded, optionally containing synthetic, non-natural or altered nucleotide bases capable of incorporation into DNA or RNA polymers.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

“Operably-linked” and “Operatively-linked” refer to the association of nucleic acid sequences on a single nucleic acid fragment so that the function of one is affected by the other. For example, a promoter is operably-linked with a coding sequence or functional RNA when it is capable of affecting the expression of that coding sequence or functional RNA (i.e., that the coding sequence or functional RNA is under the transcriptional control of the promoter). Coding sequences in sense or antisense orientation can be operably-linked to regulatory sequences. “Overexpression” refers to the level of expression in transgenic organisms that exceeds levels of expression in normal or untransformed organisms.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

“Preferred expression”, “Preferential transcription” or “preferred transcription” interchangeably refers to the expression of gene products that are preferably expressed at a higher level in one or a few plant tissues (spatial limitation) and/or to one or a few plant developmental stages (temporal limitation) while in other tissues/developmental stages there is a relatively low level of expression.

“Primary transformant” and “T0 generation” refer to transgenic plants that are of the same genetic generation as the tissue that was initially transformed (i.e., not having gone through meiosis and fertilization since transformation). “Secondary transformants” and the “T1, T2, T3, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

The terms “protein,” “peptide” and “polypeptide” are used interchangeably herein. “Promoter” or “transcription regulating nucleotide sequence” refers to a nucleotide sequence, which controls the expression of a coding sequence by providing the recognition for RNA polymerase and other factors required for proper transcription. “Promoter regulatory sequences” can comprise proximal and more distal upstream elements and/or downstream elements. Promoter regulatory sequences influence the transcription, RNA processing or stability, or translation of the associated coding sequence. Regulatory sequences include enhancers, untranslated leader sequences, introns, exons, and polyadenylation signal sequences. They include natural and synthetic sequences as well as sequences that can be a combination of synthetic and natural sequences. An “enhancer” is a nucleotide sequence that can stimulate promoter activity and can be an innate element of the promoter or a heterologous element inserted to enhance the level or tissue specificity of a promoter. The primary sequence can be present on either strand of a double-stranded DNA molecule, and is capable of functioning even when placed either upstream or downstream from the promoter. The meaning of the term “promoter” includes “promoter regulatory sequences.”

“Reference sequence” as used herein is defined as a sequence that is used as a basis for sequence comparison. A reference sequence may be a subset or the entirety of a specified sequence; for example, as a fragment of a full-length cDNA or gene sequence, or the full-length cDNA or gene sequence.

“Regulated promoter” refers to promoters that direct gene expression not constitutively, but in a temporally- and/or spatially-regulated manner, and include both tissue-specific and inducible promoters. It includes natural and synthetic sequences as well as sequences which may be a combination of synthetic and natural sequences. Different promoters may direct the expression of a gene in different tissues or cell types, or at different stages of development, or in response to different environmental conditions.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translational enhancer sequences, introns, and polyadenylation signal sequences. They include natural and synthetic sequences as well as sequences which may be a combination of synthetic and natural sequences.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived by posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA. A “functional RNA” refers to an antisense RNA, ribozyme, or other RNA that is not translated, but participates in a reaction or process as an RNA.

“Intron” refers to an intervening section of DNA that occurs almost exclusively within a eukaryotic gene, but which is not translated to amino acid sequences in the gene product. The introns are removed from the pre-mature mRNA through a process called splicing, which joins the exons to form an mRNA. For purposes of the presently disclosed subject matter, the definition of the term “intron” includes modifications to the nucleotide sequence of an intron derived from a target gene.

“Exon” refers to a section of DNA that carries the coding sequence for a protein or part of it. Exons are separated by intervening, non-coding sequences (introns). For purposes of the presently disclosed subject matter, the definition of the term “exon” includes modifications to the nucleotide sequence of an exon derived from a target gene.

A “selectable marker gene” refers to a gene whose expression in a plant cell gives the cell a selective advantage. The selective advantage possessed by the cells transformed with the selectable marker gene may be due to their ability to grow in presence of a negative selective agent, such as an antibiotic or a herbicide, compared to the ability to grow of non-transformed cells. The selective advantage possessed by the transformed cells may also be due to their enhanced capacity, relative to non-transformed cells, to utilize an added compound as a nutrient, growth factor or energy source. A selective advantage possessed by a transformed cell may also be due to the loss of a previously possessed gene in what is called “negative selection”. In this, a compound is added that is toxic only to cells that did not lose a specific gene (a negative selectable marker gene) present in the parent cell (typically a transgene).

“Specific expression” is the expression of gene products that is limited to one or a few plant tissues (spatial limitation) and/or to one or a few plant developmental stages (temporal limitation).

Substantially identical: the phrase “substantially identical,” in the context of two nucleic acid or protein sequences, refers to two or more sequences or subsequences that have at least 60%, preferably 80%, more preferably 90, even more preferably 95%, and most preferably at least 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In an especially preferred embodiment, the sequences are substantially identical over the entire length of the coding regions. Furthermore, substantially identical nucleic acid or protein sequences perform substantially the same function.

The term “transformation” refers to the transfer of a nucleic acid fragment into the genome of a host cell, resulting in genetically stable inheritance. “Transiently transformed” refers to cells in which transgenes and foreign DNA have been introduced (for example, by such methods as Agrobacterium-mediated transformation or biolistic bombardment), but not selected for stable maintenance. “Stably transformed” refers to cells that have been selected and regenerated on a selection media following transformation.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

“Transient expression” refers to expression in cells in which a virus or a transgene is introduced by viral infection or by such methods as Agrobacterium-mediated transformation, electroporation, or biolistic bombardment, but not selected for its stable maintenance. The term “translational enhancer sequence” refers to that DNA sequence portion of a gene between the promoter and coding sequence that is transcribed into RNA and is present in the fully processed mRNA upstream (5′) of the translation start codon. The translational enhancer sequence may affect processing of the primary transcript to mRNA, mRNA stability or translation efficiency.

“Vector” is defined to include, inter alia, any plasmid, cosmid, phage or Agrobacterium binary vector in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication). Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

“Visible marker” refers to a gene whose expression does not confer an advantage to a transformed cell but can be made detectable or visible. Examples of visible markers include but are not limited to Beta-glucuronidase (GUS), luciferase (LUC) and green fluorescent protein (GFP).

“Wild-type” refers to the normal gene, virus, or organism found in nature without any known mutation.

The term “plant” refers to any plant, particularly to agronomically useful plants (e.g. seed plants), and “plant cell” is a structural and physiological unit of the plant, which comprises a cell wall but may also refer to a protoplast. The plant cell may be in form of an isolated single cell or a cultured cell, or as a part of higher organized units such as for example, a plant tissue, or a plant organ differentiated into a structure that is present at any stage of a plant's development. The promoters and compositions described herein may be utilized in any plant. Examples of plants that may be utilized in contained embodiments herein include, but are not limited to, maize (corn), wheat, rice, barley, soybean, cotton, sorghum, beans in general, rape/canola, alfalfa, flax, sunflower, safflower, millet, rye, sugarcane, sugar beet, cocoa, tea, tropical sugar beet, Brassica spp., cotton, coffee, sweet potato, flax, peanut, clover; vegetables such as lettuce, tomato, cucurbits, cassaya, potato, carrot, radish, pea, lentils, cabbage, cauliflower, broccoli, Brussel sprouts, peppers, and pineapple; tree fruits such as citrus, apples, pears, peaches, apricots, walnuts, avocado, banana, and coconut; and flowers such as orchids, carnations and roses. Other plants useful in the practice of the invention include perennial grasses, such as switchgrass, prairie grasses, Indiangrass, Big bluestem grass, miscanthus and the like. It is recognized that mixtures of plants can be used.

As used herein, “plant material,” “plant part” or “plant tissue” means plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, tubers, rhizomes and the like.

Expression of novel genes that favorably affect plant water content, total water potential, osmotic potential, and turgor can enhance the ability of the plant to tolerate drought. As used herein, the terms “drought resistance” and “drought tolerance” are used to refer to a plants increased resistance or tolerance to stress induced by a reduction in water availability, as compared to normal circumstances, and the ability of the plant to function and survive in lower-water environments, and perform in a relatively superior manner.

It is recognized that many genes that improve drought resistance have complementary modes of action. Thus, combinations of these genes might have additive and/or synergistic effects in improving drought resistance in maize. Many of these genes also improve freezing tolerance (or resistance); the physical stresses incurred during freezing and drought are similar in nature and may be mitigated in similar fashion. Benefit may be conferred via constitutive or tissue-specific expression of these genes. Spatial and temporal expression patterns of these genes may enable plants to better withstand stress.

Given the overall role of water in determining yield, it is contemplated that enabling plants to utilize water more efficiently through the methods described herein, will improve overall performance even when soil water availability is not limiting. By introducing genes that improve the ability of plants to maximize water usage across a full range of stresses relating to water availability, yield stability or consistency of yield performance may be realized. It is also contemplated that methods taught herein may confirm improved protection of the plant to abiotic stress factors such as drought, heat or chill.

### I. Metabolite Profiling

Metabolite profiling was performed to identify small molecules that are modulated in response to drought stress in plants. See Examples 1-2. Techniques for metabolite profiling are well known in the art. See e.g., U.S. Pat. Nos. 7,005,255; 7,329,489; 7,433,787; 7,550,258; 7,550,260; and 7,553,616 and U.S. Published Application Nos. 20020009740; 20040146853; 20050014132; 20060134676, 20060134677; 20060134678; 20070172820; 20070172885; 20010178599; 20070026389; 20070032969; 20070288174; 20070298998; 20080124752; 20080161228; 20090017464; 20090075284; 20090093971; and 20090155826. Biomarkers of drought identified herein include molecules associated with ammonia toxicity, including elevated levels of asparagine, allantoin, and glutamine, ( ) and reduced levels of aspartate, alanine, and glutamate (data not shown). Drought conditions also produced changes consistent with increased photorespiration, such as, elevated levels of glycine and serine and decreased levels of alanine (data not shown). Sugar alcohols were also generally increased in response to drought, including galactitol galactinol, inositol, arabitol, ribitol, and proline. Any of the foregoing changes may be used as indicators of drought stress.

### II. Nucleic Acids for Improved Drought Tolerance

Nucleic acids useful in the present invention include nucleic acids encoding components of the nitrogen assimilation pathway in plants. Previously described nucleic acids and proteins have not taught how to use such molecules for enhancing drought tolerance in plants.

Specifically, when performing methods as described herein, nucleic acids useful in the invention include (a) full length or functional fragments encoding glutamine synthetase, glutamate dehydrogenase, aspartate aminotransferase, and asparagine synthetase, which may be overexpressed in a plant to thereby confer drought tolerance to the plant and/or for identification of activators of such enzymes; (b) full length or functional fragments encoding allantoinase, allantoate amidohydrolase, and ureidoglycolate amidohydrolase, which may be used for identification of inhibitors of such enzymes; and (c) inhibitory nucleic acids having homology to a nucleic acid encoding allantoinase, allantoate amidohydrolase, and ureidoglycolate amidohydrolase, which may be expressed in a plant to thereby confer drought tolerance to the plant.

The following enzymes and associated referenced accession number and sequences may find use in many in various embodiments as described herein:

Glutamine synthetase (GS, EC 6.3.1.2) catalyzes the ATP-dependent synthesis of glutamine via the condensation of ammonium and glutamate to yield glutamine, which then provides nitrogen groups, either directly or via glutamate, for the biosynthesis of all nitrogenous compounds in the plant. Higher plants have two types of GS isoenzymes that are localized in the cytosol or in the plastid/chloroplasts. Representative nucleic acids encoding cytosolic GS isoenzymes include maize GS1-1, GS1-2, GS1-3, GS1-4, and GS1-5 (GenBank Accession Nos. X65926, X65927, X65928, X65929, and NM_{—}001111827 respectively). A representative nucleic acid encoding a plastid GS isoenzyme is maize GS-2 (GenBank Accession No. X65931). A representative nucleic acid encoding a cytosolic GS isoenzyme from pea (GenBank Accession No. PEAGSCY1A).

Glutamate dehydrogenase (GDH, EC 1.4.1.3) catalyzes the synthesis of glutamate via the condensation of ammonium and alpha ketoglutarate. A representative nucleic acid encoding glutamate dehydrogenase is maize glutamate dehydrogenase (GenBank Accession Nos. gdh1:NM_{—}001111831.1).

Aspartate aminotransferase (AspAT, EC 2.6.1.1) catalyzes the conversion of oxaloacetate to aspartate. A representative nucleic acid encoding aspartate aminotransferase is maize aspartate aminotransferase (GenBank Accession Nos. NM_{—}001155533.1).

Asparagine synthetase (EC 6.3.5.4) catalyzes three distinct chemical reactions: glutamine hydrolysis to yield ammonia takes place in the N-terminal domain. The C-terminal active site mediates both the synthesis of a beta-aspartyl-AMP intermediate and its subsequent reaction with ammonia. The ammonia released is channeled to the other active site to yield asparagine. Representative nucleic acids encoding asparagine synthetase include maize asparagine synthetase 1, asparagine synthetase 2, and asparagine synthetase 3 (GenBank NM_{—}001111997, NP_{—}001131013, AsnS2:NM_{—}001137541, NP_{—}001131014, AsnS3:NM_{—}001137542, NP_{—}001131015, and AsnS4:NM_{—}001137543), respectively. Representative nucleic acids encoding asparagine synthetase 1 from Glycine max (GenBank Accession No. U77679.1).

Allantoinase (EC 3.5.2.5) catalyzes the conversion of allantoin to allantoate. Representative nucleic acids encoding allantoinase include maize allantoinase (GenBank Accession No. NM_{—}001148584, EU973141, NP_{—}001142056) respectively. Rice allantoinase (GenBank Accession No. NM_{—}001060821). Arabidopsis thaliana allantoinase (GenBank Accession No. NP_{—}567276). A Robinia pseudocacia allantoinase nucleic acid (GenBank Accession No. AY466437). A Saccharomyces cerevisiae allantoinase nucleic acid (GenBank Accession No. YSCDAL1A).

Allantoate Amidohydrolase (EC 3.5.3.9, allantoate deiminase) catalyzes the conversion of allantoate to ureidoglycine. Representative nucleic acids encoding allantoate amidohydrolase include maize allantoate amidohydrolase (GenBank Accession No. NM_{—}001157773), soybean allantoate amidohydrolase (GenBank Accession No. FJ796239), and Arabidopsis thaliana allantoate amidohydrolase (GenBank Accession Nos. NM_{—}118126 and NP_{—}193740.1), respectively.

Ureidoglycolate Amidohydrolase (EC 3.5.3.19) catalyzes the conversion of ureidoglycolate to glycoxylate. Representative nucleic acids encoding ureidoglycolate amidohydrolase include Arabidopsis thaliana ureidoglycolate amidohydrolase (GenBank Accession No. NM_{—}123726.3 and NC_{—}003076) and Baker's yeast ureidoglycolate amidohydrolase (GenBank Accession No. UAH: NP_{—}012298, respectively).

Nucleic acid variants of the above-identified sequences, which are useful in the methods of the present invention, are described herein below.

II.A. Nucleic Acid Variants

Nucleic acids are deoxyribonucleotides or ribonucleotides and polymers thereof in single-stranded, double-stranded, or triplexed form. Unless specifically limited, the term encompasses nucleic acids containing known analogues of natural nucleotides that have similar properties as the reference natural nucleic acid. The terms nucleic acid molecule or nucleic acid may also be used in place of gene, cDNA, mRNA, or cRNA. Nucleic acids may be synthesized, or may be derived from any biological source, including any organism.

Substantially identical nucleic acids are also identified as nucleic acids that hybridize specifically to or hybridize substantially to the full length of any one of sequences disclosed herein under stringent conditions. In the context of nucleic acid hybridization, two nucleic acid sequences being compared may be designated a probe and a target. A probe is a reference nucleic acid molecule, and a target is a test nucleic acid molecule, often found within a heterogeneous population of nucleic acid molecules. A target sequence is synonymous with a test sequence.

A particular nucleotide sequence employed for hybridization studies or assays includes probe sequences that are complementary to at least an about 14 to 40 nucleotide sequence of a nucleic acid molecule of the present invention. For example, probes may comprise 14 to 20 nucleotides, or even longer where desired, such as 30, 40, 50, 60, 100, 200, 300, or 500 nucleotides or up to the full length of any one sequence as referenced herein. Such fragments may be readily prepared, for example by chemical synthesis of the fragment, by application of nucleic acid amplification technology, or by introducing selected sequences into vectors for recombinant production.

Specific hybridization refers to the binding, duplexing, or hybridizing of a molecule only to a particular nucleotide sequence under stringent conditions when that sequence is present in a complex nucleic acid mixture (e.g., total cellular DNA or RNA). Specific hybridization may accommodate mismatches between the probe and the target sequence depending on the stringency of the hybridization conditions.

Stringent hybridization conditions and stringent hybridization wash conditions in the context of nucleic acid hybridization experiments such as Southern and Northern blot analysis are both sequence-dependent and environment-dependent. Longer sequences hybridize specifically at higher temperatures. An extensive guide to the hybridization of nucleic acids is found in Tijssen, Laboratory Techniques in Biochemistry and Molecular Biology-Hybridization with Nucleic Acid Probes, 1993, part I chapter 2, Elsevier, New York, N.Y. Generally, highly stringent hybridization and wash conditions are selected to be about 5° C. lower than the thermal melting point (Tm) for the specific sequence at a defined ionic strength and pH. Typically, under stringent conditions a probe will hybridize specifically to its target subsequence, but to no other sequences.

The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook et al., eds., Molecular Cloning: A Laboratory Manual, 1989, Cold Spring Harbor Laboratory Press, Cold Spring Harbor, N.Y., for a description of SSC buffer. Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4× to 6×SSC at 40° C. For short probes (e.g., about 10 to 50 nucleotides), stringent conditions typically involve salt concentrations of less than about 1M Na+ ion, typically about 0.01 to 1M Na⁺ ion concentration (or other salts) at pH 7.0-8.3, and the temperature is typically at least about 30° C. Stringent conditions may also be achieved with the addition of destabilizing agents such as formamide. In general, a signal to noise ratio of 2-fold (or higher) than that observed for an unrelated probe in the particular hybridization assay indicates detection of specific hybridization.

The following are examples of hybridization and wash conditions that may be used to identify nucleotide sequences that are substantially identical to reference nucleotide sequences of the present invention: a probe nucleotide sequence hybridizes to a target nucleotide sequence in 7% sodium dodecyl sulphate (SDS), 0.5M NaPO₄, 1 mM EDTA at 50° C. followed by washing in 2×SSC, 0.1% SDS at 50° C.; such as, a probe and target sequence hybridize in 7% sodium dodecyl sulphate (SDS), 0.5M NaPO₄, 1 mM EDTA at 50° C. followed by washing in 1×SSC, 0.1% SDS at 50° C.; such as, a probe and target sequence hybridize in 7% sodium dodecyl sulphate (SDS), 0.5M NaPO4, 1 mM EDTA at 50° C. followed by washing in 0.5×SSC, 0.1% SDS at 50° C.; such as, a probe and target sequence hybridize in 7% sodium dodecyl sulphate (SDS), 0.5M NaPO4, 1 mM EDTA at 50° C. followed by washing in 0.1×SSC, 0.1% SDS at 50° C.; such as, a probe and target sequence hybridize in 7% sodium dodecyl sulphate (SDS), 0.5M NaPO4, 1 mM EDTA at 50° C. followed by washing in 0.1×SSC, 0.1% SDS at 65° C.; or such as, a probe and target sequence hybridize in a solution of 6×SSC (0.5% SDS) at 65° C. followed by washing in 2×SSC (0.1% SDS) and 1×SSC (0.1% SDS).

A further indication that two nucleic acid sequences are substantially identical is that proteins encoded by the nucleic acids are identical or substantially identical, share an overall three-dimensional structure, or are biologically functional equivalents, as described further herein below. Nucleic acid molecules that do not hybridize to each other under stringent conditions are still substantially identical if the corresponding proteins are substantially identical. This may occur, for example, when two nucleotide sequences encoding a same protein sequence differ as permitted by the genetic code, i.e., nucleotide sequences having degenerate codon substitutions wherein the third position of one or more selected (or all) codons is substituted with mixed-base and/or deoxyinosine residues. See Batzer et al., Nucleic Acids Res., 1991, 19:5081; Ohtsuka et al., J. Biol. Chem., 1985, 260:2605-2608; and Rossolini et al. Mol. Cell Probes, 1994, 8:91-98.

The terms identical or percent identity in the context of two or more nucleotide or protein sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term substantially identical in regards to a nucleotide or protein sequence means that a particular sequence varies from the sequence of a naturally occurring sequence by one or more deletions, substitutions, or additions, the net effect of which is to retain biological function of the nucleic acid or protein.

For comparison of two or more nucleotide or amino acid sequences, typically one sequence acts as a reference sequence to which one or more test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison may be conducted, for example, by the local homology algorithm of Smith & Waterman, Adv. Appl. Math, 1981, 2:482-489, by the homology alignment algorithm of Needleman & Wunsch, J. Mol. Biol., 1970, 48:443-453, by the search for similarity method of Pearson & Lipman, Proc. Natl. Acad. Sci. USA, 1988, 85:2444-2448, by computerized implementations of these algorithms (GAP, BESTFIT, FASTA, and TFASTA in the Wisconsin Genetics Software Package, Genetics Computer Group, Madison, Wis.), or by visual inspection. See generally, Ausubel (ed.), Short Protocols in Molecular Biology, 3rd ed., 1995, Wiley, New York.

A useful algorithm for determining percent sequence identity and sequence similarity is the BLAST algorithm, which is described in Altschul et al., J. Mol. Biol., 1990, 215:403-410. Software for performing BLAST analyses is publicly available through the National Center for Biotechnology Information (web page www.ncbi.nlm.nih.gov). The BLAST algorithm parameters determine the sensitivity and speed of the alignment. For comparison of two nucleotide sequences, the BLASTn default parameters are set at W=11 (wordlength) and E=10 (expectation), and also include use of a low-complexity filter to mask residues of the query sequence having low compositional complexity. For comparison of two amino acid sequences, the BLASTp program default parameters are set at W=3 (wordlength), E=10 (expectation), use of the BLOSUM62 scoring matrix, gap costs of existence=11 and extension=1, and use of a low-complexity filter to mask residues of the query sequence having low compositional complexity.

Nucleic acids useful in the invention also can comprise a mutagenized nucleotide sequence, including sequences comprising silent mutations. A mutation may comprise one or more residue changes, a deletion of one or more residues, or an insertion of one or more additional residues.

For example, nucleic acids useful in the invention also comprise nucleic acids of any one of the sequences referenced herein, which have been altered for expression in the host organism to account for differences in codon usage. For example, the specific codon usage in plants differs from the specific codon usage in certain microorganisms. Comparison of the usage of codons within a cloned microbial open reading frame to usage in plant genes (and in particular genes from the target plant) will enable an identification of the codons within the open reading frame that should specifically be changed. Typically plant evolution has tended towards a strong preference of the nucleotides C and G in the third base position of monocotyledons, whereas dicotyledons often use the nucleotides A or T at this position. Plant genes typically have a GC content of more than 35%. Open reading frame sequences which are rich in A and T nucleotides can cause several problems in plants. Firstly, motifs of ATTTA are believed to cause destabilization of messages and are found at the 3′ end of many short-lived mRNAs. Secondly, the occurrence of polyadenylation signals such as AATAAA at inappropriate positions within the message is believed to cause premature truncation of transcription. In addition, monocotyledons may recognize AT-rich sequences as splice sites. By modifying a gene to incorporate specific codon usage for a particular target transgenic species, problems associated with GC/AT content and illegitimate splicing will be overcome.

Nucleic acids useful in the invention also include nucleic acids complementary to any sequence referenced herein or variants thereof as described herein. Complementary sequences are two nucleotide sequences that comprise antiparallel nucleotide sequences capable of pairing with one another upon formation of hydrogen bonds between base pairs. As used herein, the term complementary sequences means nucleotide sequences which are substantially complementary, as may be assessed by the same nucleotide comparison methods set forth below, or is defined as being capable of hybridizing to the nucleic acid segment in question under relatively stringent conditions such as those described herein. A particular example of a complementary nucleic acid segment is an antisense oligonucleotide.

When preparing any of the foregoing nucleic acid variants encoding a functional glutamine synthetase, glutamate dehydrogenase, aspartate aminotransferase, or asparagine synthetase enzyme, it is expected that conservative amino acid changes introduced in identifiable functional domains of such enzymes would not result in a change of enzymatic activity. Such identifiable functional domains are summarized below with respect to representative sequences. By performing routine alignments between the disclosed representative sequences and variant sequences described herein, one skilled in the art could readily identify corresponding functional domains in the variant sequences as well. Likewise, it is expected that conservative or non-conservative amino acid substitutions introduced outside of such domains would also not result in a change of enzymatic activity.

A conservatively substituted variant refers to a polypeptide comprising an amino acid sequence in which one or more residues have been conservatively substituted with a functionally similar residue. Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

Glutamine synthetase possess a beta Grasp domain having the consensus sequence [FYWL]-D-G-S-S-x(6,8)-[DENQSTAK]-[SA]-[DE]-x(2)-[LIVMFY], and ATP binding domain, and a catalytic glutamate-ammonia ligase activity domain. The locations of the beta-Grasp domain and catalytic domains are exemplified by GS1-5, GS1-1, and GS2. For example, GS1-5 has a beta-Grasp domain (this binding site is referred to on PFAM as pfam03951) that includes amino acids 17-97 and a catalytic domain that includes amino acids 103-354 (pfam00120). GS1-1 has a beta-Grasp domain (pfam03951) that includes amino acids 68-148 and a catalytic domain that includes amino acids 154-405. GS2 has a beta-Grasp domain (pfam03951) that includes amino acids 17-97 and a catalytic domain that includes amino acids 103-353. The crystal structure of GS has been described, which can also be used to identify residues that may be changed while preserving the enzyme structure and function. See Unno et al., J. Biol. Chem., 2006, 281(39):29287-29296.

Glutamate dehydrogenase contains an ELFV_dehydrogen_N domain that includes amino acids 31-161; a NADP binding domain that includes amino acids 176-402; and a NADP binding site that includes amino acids 215-217, 237-238, 289-290, and 310-312. NAD binding involves numerous hydrogen-bonds and van der Waals contacts, in particular H-bonding of residues in a turn between the first strand and the subsequent helix of the Rossmann-fold topology. Characteristically, this turn exhibits a consensus binding pattern similar to GXGXXG, in which the first 2 glycines participate in NAD(P)-binding, and the third facilitates close packing of the helix to the beta-strand. Glutamate dehydrogenase may contain a second domain in addition to the NADP domain, which is responsible for specifically binding a substrate and catalyzing a particular enzymatic reaction.

Aspartate aminotransferase is a pyridoxal phosphate (PLP)-dependent enzyme of fold type I having a pyridoxal 5′-phosphate (PLP) binding site that includes amino acids 159-161, 187, 240, 271, 301, 303-304, and 312; an AAT_like region that includes amino acids 86-451; a homodimer interface that includes amino acids 162, 197, 264, 310-312, 348, and 351; and a catalytic residue that includes amino acid 304.

Asparagine synthetase 1 from corn has an active site that includes amino acids 2, 50, 75-77, and 98; a dimer interface site that includes amino acids 17, 25, 28, 32-36, 49; a ligand binding site that includes amino acids 231-233, 265-267, 327, 341-343; and a molecular tunnel that includes amino acids 231-233, 265-267, 327, 341-343.

Asparagine synthetase 2 has an active site that includes amino acids 2, 52, 77-79, an 102; a dimer interface site that includes amino acids 20, 28, 31, 34-38, and 51; a ligand binding site that includes amino acids 248-250, 282-284, 344, and 358-360; and a molecular tunnel that includes amino acids 248-250, 282-284, 344, and 358-360.

Asparagine synthetase 3 has an active site that includes amino acids 2, 50, 75-77 and 99; a dimer interface site that includes amino acids 18, 26, 29, 32-36, and 49; a ligand binding site that includes amino acids 232-234, 266-268, 328, 342-344; and a molecular tunnel that includes amino acids 232-234, 266-268, 328, and 342-344.

Asparagine synthetase 4 has an active site that includes amino acids 2, 50, 75-77, and 99; a dimer interface site that includes amino acids 18, 26, 29, 32-36, and 49; a ligand binding site that includes amino acids 232-234, 266-268, 328, and 342-344; and a molecular tunnel that includes amino acids 232-234, 266-268, 328, and 342-344.

Nucleic acids as described herein may be cloned, synthesized, altered, mutagenized, or combinations thereof. Standard recombinant DNA and molecular cloning techniques used to isolate nucleic acids are known in the art. Site-specific mutagenesis to create base pair changes, deletions, or small insertions is also known in the art. See e.g., Sambrook et al. (eds.) Molecular Cloning: A Laboratory Manual, 1989, Cold Spring Harbor Laboratory Press, Cold Spring Harbor, N.Y.; Silhavy et al., Experiments with Gene Fusions, 1984, Cold Spring Harbor Laboratory Press, Cold Spring Harbor, N.Y.; Glover & Hames, DNA Cloning: A Practical Approach, 2nd ed., 1995, IRL Press at Oxford University Press, Oxford/N.Y.; Ausubel (ed.) Short Protocols in Molecular Biology, 3rd ed., 1995, Wiley, New York.

II.B. Nucleic Acids for Inhibition of Expression

The present invention also provides inhibitory nucleic acids having homology to nucleic acids encoding allantoinase, allantoate amidohydrolase, and ureidoglycolate amidohydrolase, which are the targets for inhibition. Inhibitory nucleic acids are well-known in the art, including nucleic acids for cosuppression, antisense inhibition, viral-suppression, hairpin suppression, stem-loop suppression, double-stranded RNA-inhibition, and interfering RNAs (e.g., short interfering RNAs (siRNA) and microinterfereing RNAs (miRNA)).

Antisense inhibition refers to the production of antisense RNA transcripts capable of suppressing the expression of the target gene or gene product. Antisense RNA refers to an RNA transcript that is complementary to all or part of a target primary transcript or mRNA and that blocks the expression of a target isolated nucleic acid fragment (U.S. Pat. No. 5,107,065). The complementarities of an antisense RNA may be with any part of the specific gene transcript, i.e., at the 5′ non-coding sequence, 3′ non-coding sequence, introns, or the coding sequence.

Cosuppression refers to the production of sense RNA transcripts capable of suppressing the expression of the target gene or gene product. Sense RNA is an RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence. See e.g., Vaucheret et al., Plant J., 1998, 16:651-659 and Gura, Nature, 2000, 404:804-808.

Another variation describes the use of plant viral sequences to direct the suppression of proximal mRNA encoding sequences. See e.g., PCT International Publication No. WO 98/36083.

Inhibitory nucleic acids may comprise hairpin structures that incorporate all, or part, of an mRNA encoding sequence in a complementary orientation that results in a potential stem-loop structure for the expressed RNA. See e.g., PCT International Publication No. WO 99/53050. In this case, the stem is formed by polynucleotides having homology to the target sequence inserted in either sense or anti-sense orientation with respect to the promoter, and the loop is formed by polynucleotides that do not have a complement in the construct. Hairpin structures may increase the frequency of cosuppression or silencing in the recovered transgenic plants. For a review of hairpin suppression, see Wesley et al., Methods in Molecular Biology, Plant Functional Genomics: Methods and Protocols, 2003, 236:273-286 and PCT International Publication Nos. WO 99/61632, WO 02/00894, WO 02/00904.

RNA interference refers to the process of sequence-specific post-transcriptional gene silencing in animals mediated by short interfering RNAs (siRNAs). See e.g., Fire et al., Nature, 1998, 391:806. The corresponding process in plants is commonly referred to as post-transcriptional gene silencing (PTGS) or RNA silencing. The presence of long dsRNAs in cells stimulates the activity of a ribonuclease III enzyme referred to as dicer. Dicer is involved in the processing of the dsRNA into short pieces of dsRNA known as short interfering RNAs (siRNAs) (Berstein et al., Nature, 2001, 409:363). Short interfering RNAs derived from dicer activity are typically about 21 to about 23 nucleotides in length and comprise about 19 base pair duplexes (Elbashir et al., Genes Dev., 2001, 15:188). Dicer has also been implicated in the excision of 21- and 22-nucleotide small temporal RNAs (stRNAs) from precursor RNA of conserved structure that are implicated in translational control (Hutvagner et al., Science, 2001, 293:834 (2001)). The RNAi response also features an endonuclease complex, commonly referred to as an RNA-induced silencing complex (RISC), which mediates cleavage of single-stranded RNA having sequence complementarities to the antisense strand of the siRNA duplex. Cleavage of the target RNA takes place in the middle of the region complementary to the antisense strand of the siRNA duplex. In addition, RNA interference can also involve small RNA (e.g., miRNA) mediated gene silencing, presumably through cellular mechanisms that regulate chromatin structure and thereby prevent transcription of target gene sequences. See, e.g., Allshire, Science, 2002, 297:1818-1819; Volpe et al., Science, 2002, 297:1833-1837; Jenuwein, Science, 2002, 297:2215-2218; and Hall et al., Science, 2002, 297:2232-2237.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

Sequence complementarities between small RNAs and their RNA targets may determine which mechanism, RNA cleavage or translational inhibition, is employed. While not intending to be limited by a particular mode of action, perfect or near-perfect complementary siRNAs are thought to mediate RNA cleavage, whereas miRNA/target duplexes containing many mismatches may mediate translational inhibition.

MicroRNAs (miRNAs) are noncoding RNAs of about 19 to about 24 nucleotides, which are processed from longer precursor transcripts that range in size from approximately 70 to 200 nt. See e.g., Lagos-Quintana et al., Science, 2001, 294:853-858, Lagos-Quintana et al., Curr. Biol., 2002, 12:735-739; Lau et al., Science, 2001, 294:858-862; Lee et al., Science, 2001, 294:862-864; Llave et al., Plant Cell, 2002, 14:1605-1619 (2002); Mourelatos et al., Genes Dev., 2002, 16:720-728; Park et al., Curr. Biol., 2002, 12:1484-1495; Reinhart et al., Genes Dev., 2002, 16:1616-1626. Processing of miRNA precursors is mediated by DCL1 (previously named CARPEL FACTORY/SHORT INTEGUMENTS1/SUSPENSOR1). See e.g., Park et al., Curr. Biol., 2002, 12:1484-1495; Reinhart et al., Genes Dev., 2002, 16:1616-1626. MicroRNAs (miRNAs) appear to regulate target genes by binding to complementary sequences located in the transcripts produced by these genes. Binding of miRNA may cause downregulation of steady-state levels of the protein encoded by the target mRNA without affecting the transcript itself (see e.g., Olsen and Ambros, Dev. Biol., 1999, 216:671-610) or may cause specific RNA cleavage of the target transcript within the target site (see e.g., Hutvagner et al., Science, 2002, 297:2056-2060; Llave et al., Plant Cell, 2002, 14:1605-1619). Accordingly, it appears that miRNAs can enter at least two pathways of target gene regulation: (1) protein downregulation when target complementarities is <100%; and (2) RNA cleavage when target complementarities is 100%. MicroRNAs entering the RNA cleavage pathway are analogous to the 21-25 nucleotide short interfering RNAs (siRNAs) generated during posttranscriptional gene silencing (PTGS).

Inhibitory nucleic acid molecules as described herein above can be used to mediate gene silencing via interaction with RNA transcripts or alternately by interaction with particular gene sequences, wherein such interaction results in gene silencing either at the transcriptional or post-transcriptional level. Inhibitory nucleic acids useful in the invention may have perfect or near-perfect complementarities with a target allantoinase, allantoate amidohydrolase, and ureidoglycolate amidohydrolase molecule, or may have several mismatches, including any of the variations described herein above under the heading “Nucleic Acid Variants.” Inhibitory nucleic acids may be expressed in plants in the same manner as for coding sequences, as described further below.

II.C. Expression Constructs

A construct for expression of any of the nucleic acids useful in the invention may include a vector sequence comprising a nucleic acid to be expressed operably linked to a promoter. A construct for recombinant expression may also comprise transcription termination signals and sequences required for proper translation of the nucleic acid. Preparation of an expression construct, including addition of translation and termination signal sequences, is known to one skilled in the art.

The promoter may comprise any polynucleotide sequence which shows transcriptional activity in the chosen plant cells, plant parts, or plants. The promoter may be native or analogous, or foreign or heterologous, to the plant host and/or to the DNA sequence of the invention. Where the promoter is native or endogenous to the plant host, it is intended that the promoter is found in the native plant into which the promoter is introduced. Where the promoter is foreign or heterologous to the DNA sequence of the invention, the promoter is not the native or naturally occurring promoter for the operably linked nucleic acid of the invention. The promoter may be inducible or constitutive. It may be naturally-occurring, may be composed of portions of various naturally-occurring promoters, and may be partially or totally synthetic. Guidance for the design of promoters is provided by studies of promoter structure, such as that of Harley et al., Nucleic Acids Res., 1987, 15:2343-61. Also, the location of the promoter relative to the transcription start may be optimized. See e.g., Roberts et al., Proc. Natl. Acad. Sci. USA, 1979, 76:760-4. Many suitable promoters for use in plants are well known in the art.

For example, suitable constitutive promoters for use in plants include the promoters from plant viruses, such as the Cestrum Virus Promoter (Stovolone et al., Plant Mol. Biol., 2003, 53(5):663-673, peanut chlorotic streak caulimovirus (PClSV) promoter (U.S. Pat. No. 5,850,019); the 35S promoter from cauliflower mosaic virus (CaMV) (Odell et al., Nature, 1985, 313:810-812); promoters of Chlorella virus methyltransferase genes (U.S. Pat. No. 5,563,328) and the full-length transcript promoter from figwort mosaic virus (FMV) (U.S. Pat. No. 5,378,619); the promoters from such genes as rice actin (McElroy et al. (1990) Plant Cell 2:163-171); ubiquitin (Christensen et al., Plant Mol. Biol., 1989, 12:619-632 and Christensen et al., Plant Mol. Biol., 1992, 18:675-689); pEMU (Last et al., Theor. Appl. Genet., 1991, 81:581-588); MAS (Velten et al., EMBO J., 1984, 3:2723-2730); maize H3 histone (Lepetit et al., Mol. Gen. Genet., 1992, 231:276-285 and Atanassova et al., Plant J., 1992, 2(3):291-300); Brassica napus ALS3 (PCT International Publication No. WO 97/41228); and promoters of various Agrobacterium genes (see U.S. Pat. Nos. 4,771,002; 5,102,796; 5,182,200; and 5,428,147).

Suitable inducible promoters for use in expressing the above-described nucleic acids in a plant include promoters that are induced by drought or by elevated levels of ammonia. For example, the DREB2A and DREB2B promoters are induced by dehydration (Liu et al., Plant Cell 1998, 10(8):1391-1406). The dehydration-responsive element (DRE) with the core sequence A/GCCGAC was identified as a cis-acting promoter element that regulates gene expression in response to drought, salt, and cold stresses in Arabidopsis (Sakuma et al., Proc. Natl. Acad. Sci. USA, 2006, 103:18822-18827). The soybean cytosolic glutamine synthase (GS) gene promoter is induced by ammonia. See Miao et al., Plant Cell, 1991, 3(1):11-22. In preferred embodiments a zea mays RAB 17 promoter which is induced by ABA as described in Busk et al. 1997, The Plant Journal 11(6), pp. 1285-1295 herein incorporated by reference, may be used in conjunction with the embodiments disclosed herein. Additionally drought inducible promoters as disclosed in U.S. Pat. No. 7,314,757 B2 may be used in certain aspects of the invention as disclosed herein.

Other inducible promoters for use in plants include those that respond to an inducing agent to which plants do not normally respond, i.e., a chemical or ligand inducer. An exemplary inducible promoter of this type is the inducible promoter from a steroid hormone gene, the transcriptional activity of which is induced by a glucocorticosteroid hormone (Schena et al., Proc. Natl. Acad. Sci. USA, 1991, 88:10421) or the recent application of a chimeric transcription activator, XVE, for use in an estrogen receptor-based inducible plant expression system activated by estradiol (Zuo et al., Plant J., 2000, 24:265-273).

Additional inducible promoters that may be used include the promoter from the ACE1 system which responds to copper (Mett et al., Proc. Natl. Acad. Sci. USA, 1993, 90:4567-4571); the promoter of the maize In2 gene which responds to benzenesulfonamide herbicide safeners (Hershey et al., Mol. Gen. Genetics, 1991, 227:229-237 and Gatz et al., Mol. Gen. Genetics, 1994, 243:32-38); and the promoter of the Tet repressor from Tn10 (Gatz et al., Mol. Gen. Genet., 1991, 227:229-237). Still other inducible promoters for use in plants are described in EP 332104, PCT International Publication Nos. WO 93/21334 and WO 97/06269. Promoters composed of portions of other promoters and partially or totally synthetic promoters can also be used. See e.g., Ni et al., Plant J., 1995, 7:661-616 and PCT International Publication No. WO 95/14098 describing such promoters for use in plants.

The promoter may include, or be modified to include, one or more enhancer elements to thereby provide for higher levels of transcription. Suitable enhancer elements for use in plants include the PClSV enhancer element (U.S. Pat. No. 5,850,019), the CaMV 35S enhancer element (U.S. Pat. Nos. 5,106,739 and 5,164,316) and the FMV enhancer element (Maiti et al., Transgenic Res., 1997, 6:143-156). See also PCT International Publication No. WO 96/23898.

Such constructs can contain a ‘signal sequence’ or ‘leader sequence’ to facilitate co-translational or post-translational transport of the peptide of interest to certain intracellular structures such as the chloroplast (or other plastid), endoplasmic reticulum, or Golgi apparatus, or to be secreted. For example, the construct can be engineered to contain a signal peptide to facilitate transfer of the peptide to the endoplasmic reticulum. A signal sequence is known or suspected to result in cotranslational or post-translational peptide transport across the cell membrane. In eukaryotes, this typically involves secretion into the Golgi apparatus, with some resulting glycosylation. A leader sequence refers to any sequence that, when translated, results in an amino acid sequence sufficient to trigger co-translational transport of the peptide chain to a sub-cellular organelle. Thus, this includes leader sequences targeting transport and/or glycosylation by passage into the endoplasmic reticulum, passage to vacuoles, plastids including chloroplasts, mitochondria, and the like. Plant expression cassettes may also contain an intron, such that mRNA processing of the intron is required for expression.

Specific initiation signals may also be used to achieve more efficient translation of sequences encoding the polypeptide of interest. Such signals include the ATG initiation codon and adjacent sequences. In cases where sequences encoding the polypeptide of interest, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers that are appropriate for the particular cell system that is used, such as those described in the literature (Scharf et al., Results Probl. Cell Differ., 1994, 20:125).

Such constructs can also contain 5′ and 3′ untranslated regions. A 3′ untranslated region is a polynucleotide located downstream of a coding sequence. Polyadenylation signal sequences and other sequences encoding regulatory signals capable of affecting the addition of polyadenylic acid tracts to the 3′ end of the mRNA precursor are 3′ untranslated regions. A 5′ untranslated region is a polynucleotide located upstream of a coding sequence.

The termination region may be native with the transcriptional initiation region, may be native with the sequence of the present invention, or may be derived from another source. Convenient termination regions are available from the Ti-plasmid of A. tumefaciens, such as the octopine synthase and nopaline synthase termination regions, or the termination region of a plant gene, such as soybean storage protein. See also Guerineau et al., Mol. Gen. Genet., 1991, 262:141-144; Proudfoot, Cell, 1991, 64:671-614; Sanfacon et al., Genes Dev. 1991, 5:141-149; Mogen et al., Plant Cell, 1990, 2:1261-1272; Munroe et al., Gene, 1990, 91:151-158; Ballas et al., Nucleic Acids Res., 1989, 17:7891-7903; and Joshi et al., Nucleic Acid Res., 1987, 15:9627-9639.

Where appropriate, the vector and sequence to be expressed may be optimized for increased expression in the transformed host cell. That is, the sequences can be synthesized using host cell-preferred codons for improved expression, or may be synthesized using codons at a host-preferred codon usage frequency. Generally, the GC content of the polynucleotide will be increased. See e.g., Campbell et al., Plant Physiol., 1990, 92:1-11 for a discussion of host-preferred codon usage. Methods are known in the art for synthesizing host-preferred polynucleotides. See e.g., U.S. Pat. Nos. 6,320,100; 6,075,185; 5,380,831; and 5,436,391, U.S. Published Application Nos. 20040005600 and 20010003849, and Murray et al., Nucleic Acids Res., 1989, 17:477-498, herein incorporated by reference.

For example, polynucleotides of interest can be targeted to the chloroplast for expression. In this manner, where the polynucleotide of interest is not directly inserted into the chloroplast, the expression cassette may additionally contain a polynucleotide encoding a transit peptide to direct the nucleotide of interest to the chloroplasts. Such transit peptides are known in the art. See e.g., Von Heijne et al., Plant Mol. Biol. Rep., 1991, 9:104-126; Clark et al. J. Biol. Chem., 1989, 264:17544-17550; Della-Cioppa et al., Plant Physiol., 1987, 84:965-968; Romer et al., Biochem. Biophys. Res. Commun., 1993, 196:1414-1421; and Shah et al., Science, 1986, 233:478-481. The polynucleotides of interest to be targeted to the chloroplast may be optimized for expression in the chloroplast to account for differences in codon usage between the plant nucleus and this organelle. In this manner, the polynucleotides of interest may be synthesized using chloroplast-preferred codons. See e.g., U.S. Pat. No. 5,380,831, herein incorporated by reference.

A plant expression cassette can be inserted into a plant transformation vector, which allows for the transformation of DNA into a cell. Such a molecule may consist of one or more expression cassettes, and may be organized into more than one vector DNA molecule. For example, binary vectors are plant transformation vectors that utilize two non-contiguous DNA vectors to encode all requisite cis- and trans-acting functions for transformation of plant cells (Hellens et al., Trends in Plant Science, 2000, 5:446-451).

A plant transformation vector comprises one or more DNA vectors for achieving plant transformation. For example, it is a common practice in the art to utilize plant transformation vectors that comprise more than one contiguous DNA segment. These vectors are often referred to in the art as binary vectors. Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a polynucleotide of interest (i.e., a polynucleotide engineered to be capable of expression in a plant cell for which generation of transgenic plants is desired). Also present in this plasmid vector are sequences required for bacterial replication. The cis-acting sequences are arranged in a fashion to allow efficient transfer into plant cells and expression therein. For example, the selectable marker sequence and the sequence of interest are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as in understood in the art (Hellens et al., 2000). Several types of Agrobacterium strains (e.g., LBA4404, GV3101, EHA101, EHA105, etc.) can be used for plant transformation. The second plasmid vector is not necessary for introduction of polynucleotides into plants by other methods such as microprojection, microinjection, electroporation, polyethylene glycol, etc.

### III. Transgenic and Knockout Plants

The present invention also provides transgenic plants comprising one or more of (1) overexpressed glutamine synthetase, glutamate dehydrogenase, aspartate aminotransferase, or asparagine synthetase nucleic acid and protein, and (2) an inhibitory nucleic acid having homology to a nucleic acid encoding allantoinase, allantoate amidohydrolase, and ureidoglycolate amidohydrolase. Also provided are knockout plants in which a genomic locus for allantoinase, allantoate amidohydrolase, and ureidoglycolate amidohydrolase is disrupted, such that transcripts encoding the enzymes are reduced or absent. The transgenic and knockout plants described herein above show increased stress tolerance as compared to wild type plants, including increased tolerance to drought and high salt concentrations. Such transgenic and knockout plants may also show increased stress tolerance to other abiotic stress conditions, such as high light intensity, low- and high-temperature, continuous light, continuous dark, and mechanical wounding.

As used herein, a plant refers to a whole plant, a plant organ (e.g., leaves, stems, roots, etc.), a seed, a plant cell, a propagule, an embryo, and progeny of the same. Plant cells can be differentiated or undifferentiated (e.g., callus, suspension culture cells, protoplasts, leaf cells, root cells, phloem cells, pollen).

Transgenic and knockout plants may be prepared in monocot or dicot plants, for example, in corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassaya, coffee, coconut, pineapple, citrus trees, cocoa, tea, banana, avocado, fig, guava, mango, olive, papaya, cashew, macadamia, almond, oats, vegetables, ornamentals, and conifers. Representative vegetables include tomatoes, lettuce, green beans, lima beans, peas, yams, onion, and members of the genus Curcumis such as cucumber, cantaloupe, and musk melon. Ornamentals include, but are not limited to, azalea, hydrangea, hibiscus, roses, tulips, daffodils, petunias, carnation, poinsettia, and chrysanthemum. The disclosed nucleic acids may also be expressed in turfgrass.

Transgenic plants, transformed plants, or stably transformed plants, or cells, tissues or seed of any of the foregoing, refer to plants that have incorporated or integrated exogenous polynucleotides into the plant cell. Stable transformation refers to introduction of a polynucleotide construct into a plant such that it integrates into the genome of the plant and is capable of being inherited by progeny thereof. For preparation of a transgenic plant as described herein, introduction of a polynucleotide, such as an expression vector, into plant cells is accomplished by one of several techniques known in the art, including but not limited to electroporation or chemical transformation See e.g., Ausubel, ed., Current Protocols in Molecular Biology, 1994, John Wiley and Sons, Inc., Indianapolis, Ind. Markers conferring resistance to toxic substances are useful in identifying transformed cells (having taken up and expressed the test polynucleotide sequence) from non-transformed cells (those not containing or not expressing the test polynucleotide sequence). Following introduction of DNA into plant cells, the transformation or integration of the polynucleotide into the plant genome is confirmed by various methods such as analysis of polynucleotides, polypeptides, and metabolites associated with the integrated sequence.

In general, plant transformation methods involve transferring heterologous DNA into target plant cells (e.g., immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent (i.e., temperature and/or herbicide). The shoots are then transferred to a selective rooting medium for recovering rooted shoot or plantlet. The transgenic plantlet then grow into mature plant and produce fertile seeds (e.g., Hiei et al., Plant J., 1994, 6:271-282; Ishida et al., Nat. Biotechnol., 1996, 14:745-750). A general description of the techniques and methods for generating transgenic plants are found in Ayres et al., CRC Crit. Rev. Plant Sci., 1994. 13:219-239, and Bommineni et al., Maydica, 1997, 42:107-120. Since the transformed material contains many cells, both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants. Then molecular and biochemical methods can be used for confirming the presence of the integrated nucleotide(s) of interest in the genome of transgenic plant. For example, selectable markers, such as, enzymes leading to changes of colors or luminescent molecules (e.g., GUS and luciferase), antibiotic-resistant genes (e.g., gentamicin and kanamycin-resistance genes) and chemical-resistant genes (e.g., herbicide-resistance genes) may be used to confirm the integration of the nucleotide(s) of interest in the genome of transgenic plant. Alternatively, considering safety of the transgenic plants, the transformed plants can be selected under environmental stresses avoiding incorporation of any selectable marker genes.

Generation of transgenic plants may be performed by one of several methods, including but not limited to introduction of heterologous DNA by Agrobacterium into plant cells (Agrobacterium-mediated transformation), bombardment of plant cells with heterologous foreign DNA adhered to particles, and various other non-particle direct-mediated methods, including microinjection, electroporation, application of Ti plasmid, Ri plasmid, or plant virus vector and direct DNA transformation (e.g., Hiei et al., Plant J., 1994, 6:271-282; Ishida et al., Nat. Biotechnol., 1996, 14:745-750; Ayres et al., CRC Crit. Rev. Plant Sci., 1994, 13:219-239; Bommineni et al., Maydica, 1997, 42:107-120) to transfer DNA.

There are three common methods to transform plant cells with Agrobacterium. The first method is co-cultivation of Agrobacterium with cultured isolated protoplasts. This method requires an established culture system that allows culturing protoplasts and plant regeneration from cultured protoplasts. The second method is transformation of cells or tissues with Agrobacterium. This method requires (a) that the plant cells or tissues can be transformed by Agrobacterium and (b) that the transformed cells or tissues can be induced to regenerate into whole plants. The third method is transformation of seeds, apices or meristems with Agrobacterium. This method requires micropropagation.

The efficiency of transformation by Agrobacterium may be enhanced by using a number of methods known in the art. For example, the inclusion of a natural wound response molecule such as acetosyringone (AS) to the Agrobacterium culture has been shown to enhance transformation efficiency with Agrobacterium tumefaciens (Shahla et al., Plant Molec. Biol, 1987, 8:291-298). Alternatively, transformation efficiency may be enhanced by wounding the target tissue to be transformed. Wounding of plant tissue may be achieved, for example, by punching, maceration, or bombardment with microprojectiles, etc. See e.g., Bidney et al., Plant Molec. Biol., 1992, 18:301-313.

In another aspect of the invention, the plant cells are transfected with vectors via particle bombardment (i.e., with a gene gun). Particle mediated gene transfer methods are known in the art, are commercially available, and include, but are not limited to, the gas driven gene delivery instrument described in U.S. Pat. No. 5,584,807, the entire contents of which are herein incorporated by reference. This method involves coating the polynucleotide sequence of interest onto heavy metal particles, and accelerating the coated particles under the pressure of compressed gas for delivery to the target tissue.

Other particle bombardment methods are also available for the introduction of heterologous polynucleotide sequences into plant cells. Generally, these methods involve depositing the polynucleotide sequence of interest upon the surface of small, dense particles of a material such as gold, platinum, or tungsten. The coated particles are themselves then coated onto either a rigid surface, such as a metal plate, or onto a carrier sheet made of a fragile material such as mylar. The coated sheet is then accelerated toward the target biological tissue. The use of the flat sheet generates a uniform spread of accelerated particles that maximizes the number of cells receiving particles under uniform conditions, resulting in the introduction of the polynucleotide sample into the target tissue.

In another aspect of the present invention, at least one genomic copy corresponding to a nucleotide sequence of the present invention is modified in the genome of the plant by homologous recombination as further illustrated in Paszkowski et al., EMBO Journal, 1988, 7:4021-26. This technique uses the property of homologous sequences to recognize each other and to exchange nucleotide sequences between each by a process known in the art as homologous recombination. Homologous recombination can occur between the chromosomal copy of a nucleotide sequence in a cell and an incoming copy of the nucleotide sequence introduced in the cell by transformation. Specific modifications are thus accurately introduced in the chromosomal copy of the nucleotide sequence. In one aspect, the regulatory elements of the nucleotide sequences of the present invention are modified. Such regulatory elements are easily obtainable by screening a genomic library using the nucleotide sequences of the present invention, or a portion thereof, as a probe. The existing regulatory elements are replaced by different regulatory elements, thus altering expression of the nucleotide sequence.

The cells that have been transformed may be grown into plants using conventional techniques. See e.g., McCormick et al., Plant Cell Rep., 1986, 5:81-84. These transgenic and knockout plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

The present invention also provides knockout plants comprising a disruption of a genomic locus encoding allantoinase, allantoate amidohydrolase, or ureidoglycolate amidohydrolase. A disrupted locus may result in expression of an altered level of the full-length enzyme encoded by the locus or expression of a mutated enzyme encoded by the locus. Plants with complete or partial functional inactivation of a locus encoding allantoinase, allantoate amidohydrolase, or ureidoglycolate amidohydrolase may be generated using standard techniques, such as T-DNA insertion or homologous recombination as described herein above. A knockout plant in accordance with the present invention may also be prepared by expression of an inhibitory nucleic acid as described herein below. The present invention also provides transgenic plants with specific “knocked-in” modifications of allantoinase, allantoate amidohydrolase, or ureidoglycolate amidohydrolase, for example to create an over-expression mutant having a dominant negative phenotype. Thus, “knocked-in” modifications include the expression of both wild type and mutated forms of a nucleic acid encoding allantoinase, allantoate amidohydrolase, or ureidoglycolate amidohydrolase.

Transgenic and knockout plants of the invention can be homozygous for the added polynucleotides; i.e., a transgenic plant that contains two added sequences, one sequence at the same locus on each chromosome of a chromosome pair. A homozygous transgenic plant can be obtained by sexually mating (selfing) an independent segregant transgenic plant that contains the added sequences according to the invention, germinating some of the seed produced and analyzing the resulting plants produced for enhanced enzyme activity (i.e., drought tolerance) and/or increased plant yield relative to a control (native, non-transgenic) or an independent segregant transgenic plant.

Two different transgenic or knockout plants can also be mated to produce offspring that contain two independently segregating added, exogenous polynucleotides. Selfing of appropriate progeny can produce plants that are homozygous for all added, exogenous polynucleotides that encode a polypeptide of the present invention. Back-crossing to a parental plant and outcrossing with a non-transgenic plant are also contemplated.

The transgenic and knockout plants having altered expression and/or activity of enzymes involved in ammonia assimilation may be further modified at second a locus to confer increased stress tolerance or other trait of interest. Representative desired traits include improved crop yield; increased seed yield; increased amino acid content; increased nitrate content; increased tolerance to stress; insect resistance; tolerance to broad-spectrum herbicides; resistance to diseases caused by viruses, bacteria, fungi, and worms; and enhancement of mechanisms for protection from environmental stresses such as heat, cold, drought, and high salt concentration. Additional desired traits include output traits that benefit consumers, for example, nutritionally enhanced foods that contain more starch or protein, more vitamins, more anti-oxidants, and/or fewer trans-fatty acids; foods with improved taste, increased shelf-life, and better ripening characteristics; trees that make it possible to produce paper with less environmental damage; nicotine-free tobacco; ornamental flowers with new colors, fragrances, and increased longevity; etc. Still further, desirable traits that may be used in accordance with the invention include gene products produced in plants as a means for manufacturing, for example, therapeutic proteins for disease treatment and vaccination; textile fibers; biodegradable plastics; oils for use in paints, detergents, and lubricants; etc. For genetic modifications that confer traits associated with altered gene expression, or increased stress tolerance, the combination of a transgenic or knockout plant as described herein and a second genetic modification can produce a synergistic effect, i.e., a change in gene expression, or increased stress tolerance that is greater than the change elicited by either genetic modification alone.

### IV. Ligands for Improved Drought Tolerance

The present invention further discloses assays to identify (1) binding partners and activators of a plant glutamine synthetase, glutamate dehydrogenase, aspartate aminotransferase, and asparagine synthetase, and (2) binding partners and inhibitors of a plant allantoinase, allantoate amidohydrolase, and ureidoglycolate amidohydrolase. Methods of identifying activators involve assaying an enhanced level or quality of enzyme expression or function in the presence of one or more test agents. Methods of identifying inhibitors involve assaying a decreased level or quality of enzyme expression or function in the presence of one or more test agents. Representative activators and inhibitors include small molecules as well as biological entities, as described herein below.

A control level or quality of enzyme activity refers to a level or quality of wild type expression activity. When evaluating the activating capacity of a test agent, a control level or quality of enzyme activity comprises a level or quality of activity in the absence of the test agent.

Significantly changed enzyme activity refers to a quantifiable change in a measurable quality that is larger than the margin of error inherent in the measurement technique. For example, significant enhancement refers to enzyme activity that is increased by about 2-fold or greater relative to a control measurement, or an about 5-fold or greater increase, or an about 10-fold or greater increase. An assay of enzyme function may comprise determining a level of enzyme expression; determining increased or decreased levels of products known to be produced by the enzyme activity; determining an active conformation of an enzyme; or determining activation of signaling events in response to an activator or inhibitor (e.g., increased drought tolerance).

In accordance with the present invention there is also provided a rapid and high throughput screening method that relies on the methods described herein. This screening method comprises separately contacting an enzyme involved in ammonia assimilation with a plurality of test agents. In such a screening method the plurality of test agents may comprise more than about 10⁴ samples, or more than about 10⁵ samples, or more than about 10⁶ samples.

The in vitro and cellular assays of the invention may comprise soluble assays, or may further comprise a solid phase substrate for immobilizing one or more components of the assay. For example, an enzyme or a cell expressing an enzyme involved in ammonia assimilation, may be bound directly to a solid state component via a covalent or non-covalent linkage. Optionally, the binding may include a linker molecule or tag that mediates indirect binding of an enzyme to a substrate.

Cellular assays, including expression assays and functional assays as described herein below, may employ a cell that endogenously expresses an enzyme involved in ammonia assimilation, a cell that expresses a heterologous nucleic acid encoding such an enzyme but otherwise lacks endogenous expression of the enzyme, or a cell that expresses endogenous enzyme as well as a heterologous nucleic acid encoding the enzyme. Representative eukaryotic host cells for recombinant expression of the nucleic acids described herein include yeast and plant cells, as well as prokaryotic hosts such as E. coli and Bacillus subtilis. A host cell may be a stable cell line. A host cell strain may be chosen which affects the expression of the recombinant sequence, or modifies and processes the gene product in a specific manner. For example, different host cells have characteristic and specific mechanisms for the translational and post-translational processing and modification (e.g., glycosylation, phosphorylation of proteins). Appropriate cell lines or host cells may be chosen to ensure the desired modification and processing of the foreign protein expressed. For example, expression in a bacterial system may be used to produce a non-glycosylated core protein product, and expression in yeast will produce a glycosylated product.

IV.A. Test Agents

A test agent refers to any agent that potentially interacts with an enzyme involved in ammonia assimilation as described herein, including any synthetic, recombinant, or natural product. A test agent suspected to interact with a protein may be evaluated for such an interaction using the methods disclosed herein.

Representative test agents include but are not limited to peptides, proteins, nucleic acids, small molecules (e.g., organic and inorganic chemical compounds), antibodies or fragments thereof, nucleic acid-protein fusions, any other affinity agent, and combinations thereof. A test agent to be tested may be a purified molecule, a homogenous sample, or a mixture of molecules or compounds.

A small molecule refers to a compound, for example an organic compound, with a molecular weight of less than about 1,000 daltons, such as less than about 750 daltons, or less than about 600 daltons, or less than about 500 daltons. A small molecule may have a computed log octanol-water partition coefficient in the range of about −4 to about +14, such as in the range of about −2 to about +7.5.

Test agents may be obtained or prepared as a library or collection of molecules. A library may contain a few or a large number of different molecules, varying from about ten molecules to several billion molecules or more. A molecule may comprise a naturally occurring molecule, a recombinant molecule, or a synthetic molecule. A plurality of test agents in a library may be assayed simultaneously. Optionally, test agents derived from different libraries may be pooled for simultaneous evaluation.

Representative libraries include but are not limited to a peptide library (U.S. Pat. Nos. 6,156,511, 6,107,059, 5,922,545, and 5,223,409), an oligomer library (U.S. Pat. Nos. 5,650,489 and 5,858,670), an aptamer library (U.S. Pat. Nos. 7,338,762; 7,329,742; 6,949,379; 6,180,348; and 5,756,291), a small molecule library (U.S. Pat. Nos. 6,168,912 and 5,738,996), a library of antibodies or antibody fragments (U.S. Pat. Nos. 6,174,708, 6,057,098, 5,922,254, 5,840,479, 5,780,225, 5,702,892, and 5,667,988), a library of nucleic acid-protein fusions (U.S. Pat. No. 6,214,553), and a library of any other affinity agent that may potentially bind to an enzyme involved in ammonia assimilation.

A library may comprise a random collection of molecules. Alternatively, a library may comprise a collection of molecules having a bias for a particular sequence, structure, or conformation, for example, as for inhibitory nucleic acids. See e.g., U.S. Pat. Nos. 5,264,563 and 5,824,483. Methods for preparing libraries containing diverse populations of various types of molecules are known in the art, for example as described in U.S. patents cited herein above. Numerous libraries are also commercially available.

IV.B. Expression Assays

In one aspect of the invention, a modulator of an enzyme involved in ammonia assimilation may be identified by assaying expression of a nucleic acid encoding the enzyme. For example, a method of identifying an agent useful for promoting drought tolerance may include the steps of (a) providing a cell expressing glutamine synthetase, glutamate dehydrogenase, aspartate aminotransferase, or asparagine synthetase; (b) contacting the cell with one or more test agents or a control agent; (c) assaying expression of a glutamine synthetase, glutamate dehydrogenase, aspartate aminotransferase, or asparagine synthetase nucleic acid or protein; and (d) selecting a test agent that induces elevated expression of the glutamine synthetase, glutamate dehydrogenase, aspartate aminotransferase, or asparagine synthetase nucleic acid or protein when contacted the cell is contacted with the test agent as compared to the control agent; whereby an agent for improving or increasing drought tolerance in a plant is identified. As another example, a method of identifying an agent useful for promoting drought tolerance may include the steps of (a) providing a cell expressing allantoinase, allantoate amidohydrolase, or ureidoglycolate amidohydrolase; (b) contacting the cell with one or more test agents or a control agent; (c) assaying expression of an allantoinase, allantoate amidohydrolase, or ureidoglycolate amidohydrolase nucleic acid or protein; and (d) selecting a test agent that induces decreased expression of the allantoinase, allantoate amidohydrolase, or ureidoglycolate amidohydrolase nucleic acid or protein when contacted the cell is contacted with the test agent as compared to the control agent; whereby an agent for improving or increasing drought tolerance in a plant is identified.

IV.C. Binding Assays

In another aspect of the invention, a method of identifying of a modulator useful for promoting drought tolerance comprises determining specific binding of a test agent to one or more of glutamine synthetase, glutamate dehydrogenase, aspartate aminotransferase, asparagine synthetase, allantoinase, allantoate amidohydrolase, and ureidoglycolate amidohydrolase. For example, a method of identifying a binding partner of one of the foregoing enzymes may comprise: (a) providing a protein; (b) contacting the protein with one or more test agents under conditions sufficient for binding; (c) assaying binding of a test agent to the isolated protein; and (d) selecting a test agent that demonstrates specific binding to the protein. Specific binding may also encompass a quality or state of mutual action such that binding of a test agent to a protein is activating or inducing. The activating nature of specific binding can be assessed by the additional assays of enzyme function described herein.

Specific binding refers to a binding reaction which is determinative of the presence of the protein in a heterogeneous population of proteins and other biological materials. The binding of a test agent to a protein may be considered specific if the binding affinity is about 1×10⁴M⁻¹ to about 1×10⁶M⁻¹ or greater. Specific binding also refers to saturable binding. To demonstrate saturable binding of a test agent to a protein, Scatchard analysis may be carried out as described, for example, by Mak et al., J. Biol. Chem., 1989, 264:21613-21618.

Several techniques may be used to detect interactions between an enzyme involved in ammonia assimilation and a test agent without employing a known competitive inhibitor. Representative methods include, but are not limited to, Fluorescence Correlation Spectroscopy, Surface-Enhanced Laser Desorption/Ionization Time-Of-flight Spectroscopy, and BIACORE® technology, each technique described herein below. These methods are amenable to automated, high-throughput screening.

Fluorescence Correlation Spectroscopy (FCS) measures the average diffusion rate of a fluorescent molecule within a small sample volume. The sample size may be as low as 10³ fluorescent molecules and the sample volume as low as the cytoplasm of a single bacterium. The diffusion rate is a function of the mass of the molecule and decreases as the mass increases. FCS may therefore be applied to protein-ligand interaction analysis by measuring the change in mass and therefore in diffusion rate of a molecule upon binding. In a typical experiment, the target to be analyzed (e.g., an enzyme involved in ammonia assimilation) is expressed as a recombinant protein with a sequence tag, such as a poly-histidine sequence, inserted at the N-terminus or C-terminus. The expression is mediated in a host cell, such as E. coli, yeast, Xenopus oocytes, or mammalian cells. The protein is purified using chromatographic methods. For example, the poly-histidine tag may be used to bind the expressed protein to a metal chelate column such as Ni2+ chelated on iminodiacetic acid agarose. The protein is then labeled with a fluorescent tag such as carboxytetramethylrhodamine or BODIPY™ reagent (available from Molecular Probes of Eugene, Oreg.). The protein is then exposed in solution to the potential ligand, and its diffusion rate is determined by FCS using instrumentation available from Carl Zeiss, Inc. (Thornwood of New York, N.Y.). Ligand binding is determined by changes in the diffusion rate of the protein.

Surface-Enhanced Laser Desorption/Ionization (SELDI) was developed by Hutchens & Yip, Rapid Commun. Mass Spectrom., 1993, 7:576-580. When coupled to a time-of-flight mass spectrometer (TOF), SELDI provides a technique to rapidly analyze molecules retained on a chip. It may be applied to ligand-protein interaction analysis by covalently binding the target protein, or portion thereof, on the chip and analyzing by mass spectrometry the small molecules that bind to this protein (Worrall et al., Anal Chem., 1998, 70(4):750-756). In a typical experiment, a target protein (e.g., an enzyme involved in ammonia assimilation) is recombinantly expressed and purified. The target protein is bound to a SELDI chip either by utilizing a poly-histidine tag or by other interaction such as ion exchange or hydrophobic interaction. A chip thus prepared is then exposed to the potential ligand via, for example, a delivery system able to pipet the ligands in a sequential manner (autosampler). The chip is then washed in solutions of increasing stringency, for example a series of washes with buffer solutions containing an increasing ionic strength. After each wash, the bound material is analyzed by submitting the chip to SELDI-TOF. Ligands that specifically bind a target protein are identified by the stringency of the wash needed to elute them.

BIACORE® relies on changes in the refractive index at the surface layer upon binding of a ligand to a target protein (e.g., an enzyme involved in ammonia assimilation) immobilized on the layer. In this system, a collection of small ligands is injected sequentially in a 2-5 microliter cell, wherein the target protein is immobilized within the cell. Binding is detected by surface plasmon resonance (SPR) by recording laser light refracting from the surface. In general, the refractive index change for a given change of mass concentration at the surface layer is practically the same for all proteins and peptides, allowing a single method to be applicable for any protein. In a typical experiment, a target protein is recombinantly expressed, purified, and bound to a BIACORE® chip. Binding may be facilitated by utilizing a poly-histidine tag or by other interaction such as ion exchange or hydrophobic interaction. A chip thus prepared is then exposed to one or more potential ligands via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics of on rate and off rate allows the discrimination between non-specific and specific interaction. See also Homola et al., Sensors and Actuators, 1999, 54:3-15 and references therein.

IV.D. Conformational Assay

The present invention also provides methods of identifying binding partners, activators, and inhibitors that rely on a conformational change of an enzyme involved in ammonia assimilation when bound by or otherwise interacting with a test agent. For example, application of circular dichroism to solutions of macromolecules reveals the conformational states of these macromolecules. The technique may distinguish random coil, alpha helix, and beta chain conformational states.

To identify binding partners, activators, and inhibitors of an enzyme involved in ammonia assimilation, circular dichroism analysis may be performed using a recombinantly expressed enzyme. An enzyme involved in ammonia assimilation is purified, for example by ion exchange and size exclusion chromatography, and mixed with a test agent. The mixture is subjected to circular dichroism. The conformation of an enzyme in the presence of a test agent is compared to a conformation of the enzyme in the absence of the test agent. A change in conformational state of the enzyme in the presence of a test agent identifies an enzyme binding partner, activator, or inhibitor. Representative methods are described in U.S. Pat. Nos. 5,776,859 and 5,780,242. Activity of the binding partner or activator may be assessed using functional assays, such assays include nitrate content, nitrate uptake, lateral root growth, seed yield, amino acid content or plant biomass, as described herein.

IV.E. Functional Assays

In another aspect of the invention, a method of identifying a modulator of an enzyme involved in ammonia assimilation employs a functional enzyme. Methods of determining enzyme activity include measuring increased or decreased levels of substrates or product of the chemical reaction known to be catalyzed by the enzyme. For example, a method of identifying an agent for improving or increasing drought tolerance in a plant can include the steps of: (a) providing a cell expressing glutamine synthetase, glutamate dehydrogenase, aspartate aminotransferase, or asparagine synthetase; (b) contacting the cell with one or more test agents or a control agent; (c) assaying a level of glutamine or asparagine; and (d) selecting a test agent that induces an elevated level of glutamine or asparagine when the cell is contacted with the test agent as compared to the control agent; whereby an agent for improving or increasing drought tolerance in a plant is identified. As another example, a method of identifying an agent for improving or increasing drought tolerance in a plant can include the steps of (a) providing a cell expressing allantoinase, allantoate amidohydrolase, or ureidoglycolate amidohydrolase; (b) contacting the cell with one or more test agents or a control agent; (c) assaying a level of allantoin; and (d) selecting a test agent that induces an increased level of allantoin when the cell is contacted with the test agent as compared to the control agent; whereby an agent for improving or increasing drought tolerance in a plant is identified.

Assays of enzyme activity that employ transiently transfected cells may include a marker that distinguishes transfected cells from non-transfected cells. A marker may be encoded by or otherwise associated with a construct for enzyme expression, such that cells are simultaneously transfected with a nucleic acid molecule encoding the enzyme and the marker. Representative detectable molecules that are useful as markers include but are not limited to a heterologous nucleic acid, a protein encoded by a transfected construct (e.g., an enzyme or a fluorescent protein), a binding protein, and an antigen. In accordance with the disclosed methods, cells expressing an enzyme involved in ammonia assimilation may be provided in the form of a kit useful for performing an assay of enzyme function. For example, a test kit for detecting an activator may include cells transfected with DNA encoding a full-length enzyme involved in ammonia assimilation and a medium for growing the cells.

Assays employing cells or plants expressing an enzyme involved in ammonia assimilation may additionally employ control cells or plants that are substantially devoid of a native enzyme involved in ammonia assimilation and, optionally, proteins substantially similar to an the enzyme. When using transiently transfected cells, a control cell may comprise, for example, an untransfected host cell. When using a stable cell line expressing an enzyme involved in ammonia assimilation, a control cell may comprise, for example, a parent cell line used to derive the enzyme-expressing cell line. When using plants, a negative control may comprise a plant transformed with a vector lacking a transgene encoding an enzyme involved in ammonia assimilation. Also when using plants, a positive control plant may include a plant overexpressing an enzyme involved in ammonia assimilation, such that an activator or inhibitor elicits a phenotype similar to an overexpressing plant.

IV.F. Rational Design

The knowledge of the structure of a native enzyme involved in ammonia assimilation provides an approach for rational design of activators and inhibitors. In brief, the structure of an enzyme may be determined by X-ray crystallography and/or by computational algorithms that generate three-dimensional representations. See Saqi et al., Bioinformatics, 1999, 15:521-522; Huang et al., Pac. Symp. Biocomput, 2000, 230-241; and PCT International Publication No. WO 99/26966. Alternatively, a working model of enzyme structure may be derived by homology modeling (Maalouf et al., J. Biomol. Struct. Dyn., 1998, 15(5):841-851). Computer models may further predict binding of a protein structure to various substrate molecules that may be synthesized and tested using the assays described herein above. Additional compound design techniques are described in U.S. Pat. Nos. 5,834,228 and 5,872,011.

The enzymes involved in ammonia assimilation disclosed herein are soluble proteins, which may be purified and concentrated for crystallization. The purified enzymes may be crystallized under varying conditions of at least one of the following: pH, buffer type, buffer concentration, salt type, polymer type, polymer concentration, other precipitating ligands, and concentration of purified enzyme. Methods for generating a crystalline protein are known in the art and may be reasonably adapted for determination of an enzyme involved in ammonia assimilation as disclosed herein. See e.g., Deisenhofer et al., J. Mol. Biol., 1984, 180:385-398; Weiss et al., FEBS Lett., 1990, 267:268-272; or the methods provided in a commercial kit, such as the CRYSTAL SCREEN™ kit (available from Hampton Research of Riverside, Calif., USA).

A crystallized enzyme may be tested for functional activity and differently sized and shaped crystals are further tested for suitability in X-ray diffraction. Generally, larger crystals provide better crystallography than smaller crystals, and thicker crystals provide better crystallography than thinner crystals. For example, enzyme crystals may range in size from 0.1-1.5 mm. These crystals diffract X-rays to at least 10 Å resolution, such as 1.5-10.0 Å or any range of value therein, such as 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5 or 3, with 3.5 Å A or less being preferred for the highest resolution.

### V. Use of Agents for Reducing Ammonia Accumulation in Plants

The disclosed binding partners, activators, and inhibitors of an enzyme involved in ammonia assimilation are useful both in vitro and in vivo for applications generally related to assessing responses to drought stress and for increasing drought tolerance. In particular, activators of glutamine synthetase, glutamate dehydrogenase, aspartate aminotransferase, and asparagine synthetase may be used to increase drought tolerance. Inhibitors of allantoinase, allantoate amidohydrolase, and ureidoglycolate amidohydrolase may similarly be used to increase drought tolerance.

Activators and inhibitors of an enzyme involved in ammonia assimilation may also be used to increase stress tolerance to other abiotic stress conditions, such as high light intensity, low- and high-temperature, continuous light, continuous dark, salinity, and mechanical wounding.

The present invention provides that an effective amount of an activator of glutamine synthetase, glutamate dehydrogenase, aspartate aminotransferase, and asparagine synthetase is applied to a plant to improve drought tolerance. An effective amount of an activator may comprise an amount sufficient to elicit elevated expression of the enzyme, elevated levels or quality of enzyme activity, and/or increased tolerance to drought conditions. The present invention further provides that an effective amount of an inhibitor of allantoinase, allantoate amidohydrolase, and ureidoglycolate amidohydrolase may be applied to a plant to improve drought tolerance. An effective amount of an inhibitor may comprise an amount sufficient to elicit reduced expression of the enzyme, reduced levels or quality of enzyme activity, and/or increased tolerance to drought conditions.

Plants that may benefit from the activators and inhibitors described herein include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassaya, coffee, coconut, pineapple, citrus trees, cocoa, tea, banana, avocado, fig, guava, mango, olive, papaya, cashew, macadamia, almond, oats, Arabidopsis thaliana, poplar, turfgrass, vegetables, ornamentals, and conifers. Representative vegetables include tomatoes, lettuce, green beans, lima beans, peas, yams, onions, and members of the genus Curcumis such as cucumber, cantaloupe, and musk melon. Representative ornamentals include, but are not limited to, azalea, hydrangea, hibiscus, roses, tulips, daffodils, petunias, carnation, poinsettia, and chrysanthemum. Any of the aforementioned plants may be wild type, inbred, or transgenic, e.g., plants strains and genetically modified plants as used in agricultural settings.

Plants treated with an activator or inhibitor of an enzyme involved in ammonia assimilation as described herein may be transgenic, i.e., genetically modified at the locus encoding the enzyme or other locus to confer increased drought tolerance or other trait of interest. Representative desired traits include improved crop yield; increased seed yield; increased amino acid content; increased nitrate content; increased tolerance to stress; insect resistance; tolerance to broad-spectrum herbicides; resistance to diseases caused by viruses, bacteria, fungi, and worms; and enhancement of mechanisms for protection from environmental stresses such as heat, cold, drought, and high salt concentration. Additional desired traits include output traits that benefit consumers, for example, nutritionally enhanced foods that contain more starch or protein, more vitamins, more anti-oxidants, and/or fewer trans-fatty acids; foods with improved taste, increased shelf-life, and better ripening characteristics; trees that make it possible to produce paper with less environmental damage; nicotine-free tobacco; ornamental flowers with new colors, fragrances, and increased longevity; etc. Still further, desirable traits that may be used in accordance with the invention include gene products produced in plants as a means for manufacturing, for example, therapeutic proteins for disease treatment and vaccination; textile fibers; biodegradable plastics; oils for use in paints, detergents, and lubricants; etc. For genetic modifications that confer traits associated with increased drought tolerance, use of an activator or inhibitor of an enzyme involved in ammonia assimilation as described herein, or overexpression of such enzymes, in combination with a first or second genetic modification, respectively, can produce a synergistic effect, i.e., a change in gene expression, or increased drought tolerance that is greater than the change elicited by the single agents/genetic modifications alone.

### EXAMPLES

The following examples have been included to illustrate modes of the invention. Certain aspects of the following examples are described in terms of techniques and procedures found or contemplated by the present co-inventors to work well in the practice of the invention. In light of the present disclosure and the general level of skill in the art, those of skill will appreciate that the following examples are intended to be exemplary only and that numerous changes, modifications, and alterations may be employed without departing from the scope of the invention. Unless indicated otherwise, The cloning steps carried out for the purposes of the present invention, such as, for example, restriction cleavages, agarose gel electrophoresis, purification of DNA fragments, transfer of nucleic acids to nitrocellulose and nylon membranes, linking DNA fragments, transformation of E. coli cells, growing bacteria, multiplying phages and sequence analysis of recombinant DNA, are carried out as described by Sambrook (1989). The sequencing of recombinant DNA molecules is carried out using ABI laser fluorescence DNA sequencer following the method of Sanger (Sanger 1977).

### Example 1

### Metabolite Profiling of Maize in Fully Irrigated or Drought Conditions

To identify metabolic changes associated with drought tolerance, six maize varieties (Entries 1-6) were grown under well-watered and drought stress conditions just prior to flowering stage. The six maize varieties were selected for study based upon lack of close genetic relation to each other. Leaf tissues were collected for metabolite profiling using methods as described in U.S. Pat. No. 7,433,787 and related methods. A high quality data set consisting of 694 metabolites was obtained. Drought stress had a significant impact on primary and secondary metabolism in the leaves. As shown in Table 1, drought stressed plants showed a significant increase in levels of asparagine, allantoin, and glutamine as compared to well watered plants. These compounds have been identified in the art to be mainly responsible for nitrogen storage and transportation in plants. Surprisingly, the most dramatic increase amongst the metabolites in drought conditions was an approximately 50 fold increase in asparagines (see Table 1). (Table 1). Entry 2 and entry 4 had the most increases of the above-noted compounds, while the changes in entry 3 and entry 6 were not apparent. In addition, in most of the lines, alanine, aspartate, and glutamate showed decreases, suggesting that the activities of asparagine synthetase and glutamine synthetase are up-regulated and allantoinase is down-regulated (see Table 1.

TABLE 1

| | Fold Change (FS/FI) | | | | | |
|---|---|---|---|---|---|---|
| | p Value | | | | | |
| Compound | Entry 1 | Entry 2 | Entry 3 | Entry 4 | Entry 5 | Entry 6 |
| aspartate | 0.29 | 0.35 | 0.34 | 0.34 | 0.59 | 0.29 |
| | 0.035 | 0.0017 | 0.0114 | 0.0128 | 0.0794 | 0.0092 |
| asparagine | 4.11 | 53.87 | 1.62 | 20.59 | 3.66 | 0.99 |
| | 0.0079 | 0.0004 | 0.1461 | 0.0071 | 0.0108 | 0.8909 |
| glutamate | 0.8 | 0.47 | 0.57 | 0.77 | 0.95 | 0.45 |
| | 0.513 | 0.0002 | 0.0049 | 0.3226 | 0.7561 | 0.0054 |
| glutamine | 1.3 | 4.76 | 0.78 | 3.79 | 2.59 | 0.77 |
| | 0.2147 | 0.0001 | 0.236 | 0.009 | 0.0007 | 0.0931 |
| allantoin | 1.69 | 36.69 | 5.92 | 7.28 | 9.85 | 2.06 |
| | 0.145 | 0.0028 | 0.0045 | 0.0033 | 0.0064 | 0.2609 |

It was also observed that the levels of most other amino acids increased under drought conditions (data not shown). Particularly, it was observed that there is an increase in glycine and serine and a decrease in alanine levels. During photorespiration, alanine is a primary amino donor to produce glycine and then serine. The increases of glycine and serine and decrease of alanine are consistent with accelerated photorespiration in response to drought stress.

In addition to amino acids, several sugar alcohols accumulated as osmolytes under drought stress (data not shown). Among the 6 entry lines, entry 6 had the least increases of the sugar alcohols. Surprisingly, the amino acid proline which has been used extensively as a classical marker for drought stress, only increased in entry 5 (p<0.05), entry 3 (p>0.05), and entry 6 (p>0.05).

### Example 2

### Metabolite Profiling of Drought Tolerant Maize in Fully Irrigated and Drought Conditions

A second metabolite profiling study was performed to detect changes in asparagine, glutamine, allantoin, and proline in different tissues and at different time points following drought stress. In this study, two corn hybrids (R and S) with contrasting drought tolerance levels were used. The corn hybrids R and S were previously characterized as drought tolerant and drought intolerant varieties, respectively. The hybrids were planted in each of two plots. One plot was used for drought treatment and the other one was used as well watered or fully irrigated condition. Each plot contained 6 subplots as replicates. Each subplot contained each of the corn hybrids and 2 replicated rows for sampling and yield measurement. Four (4) out of 6 subplots were chosen for sampling.

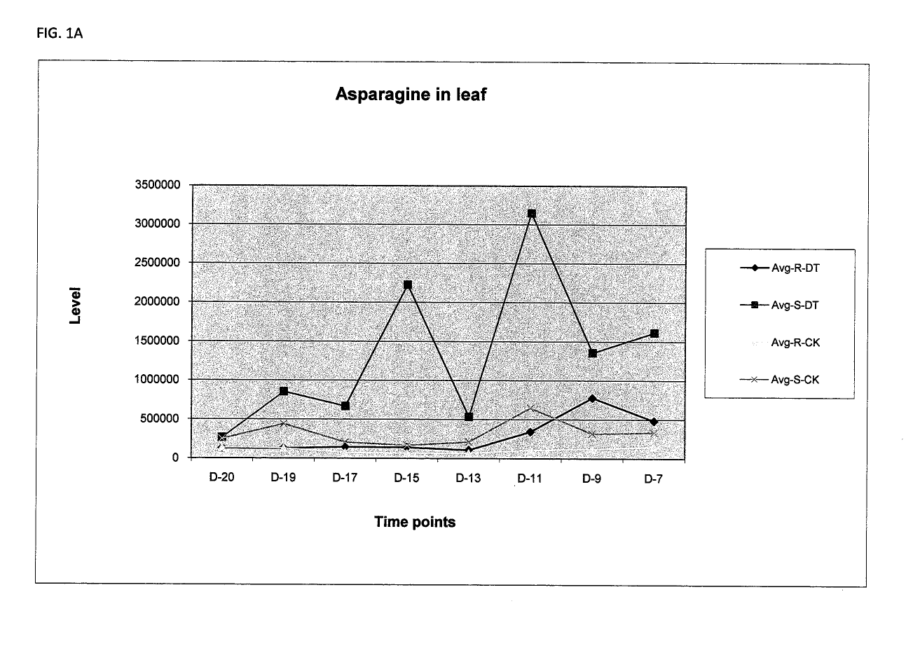

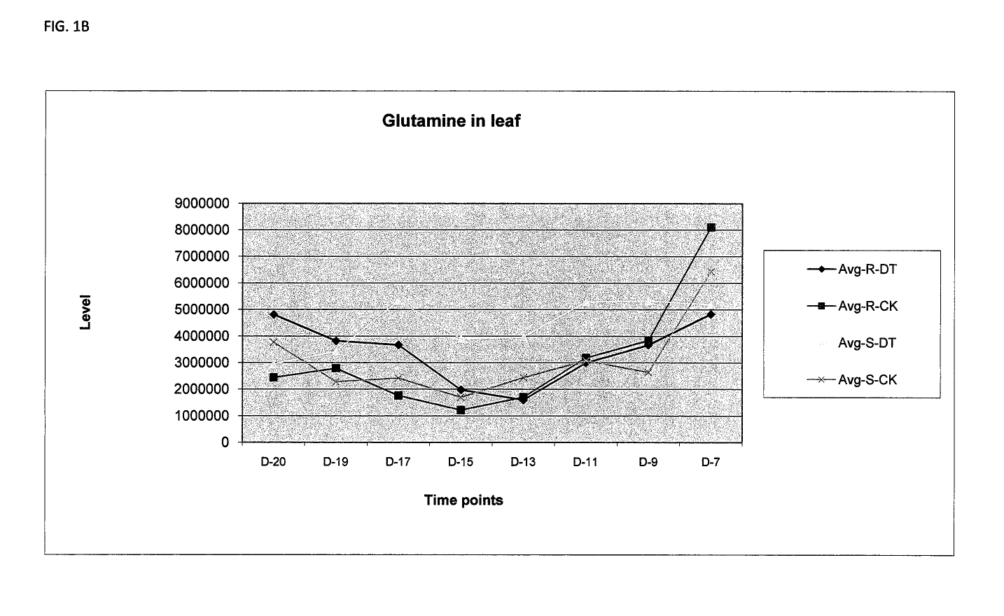

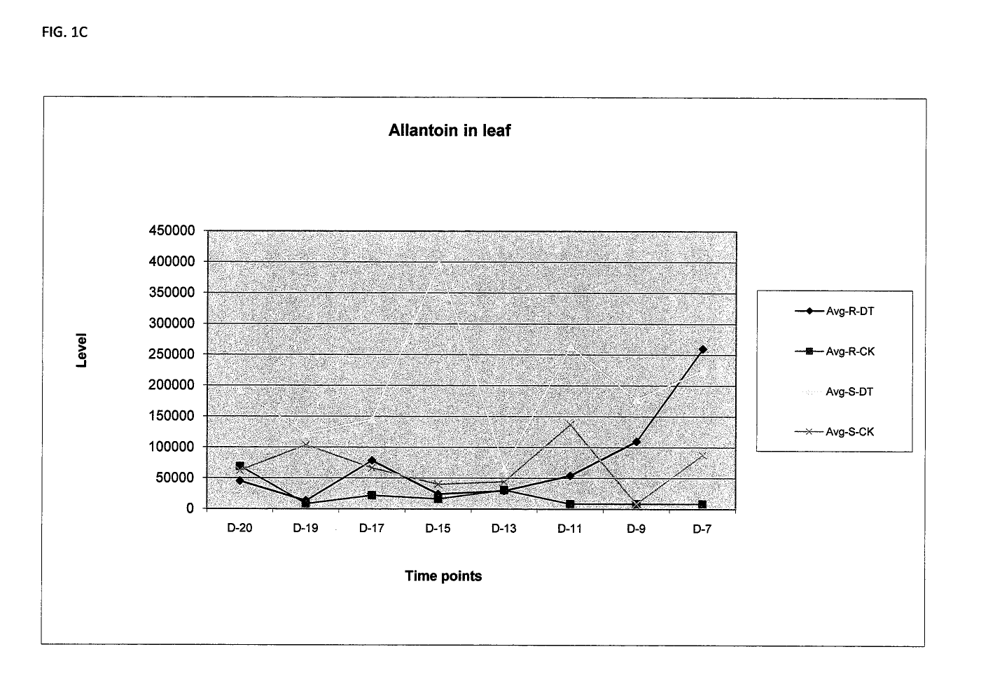

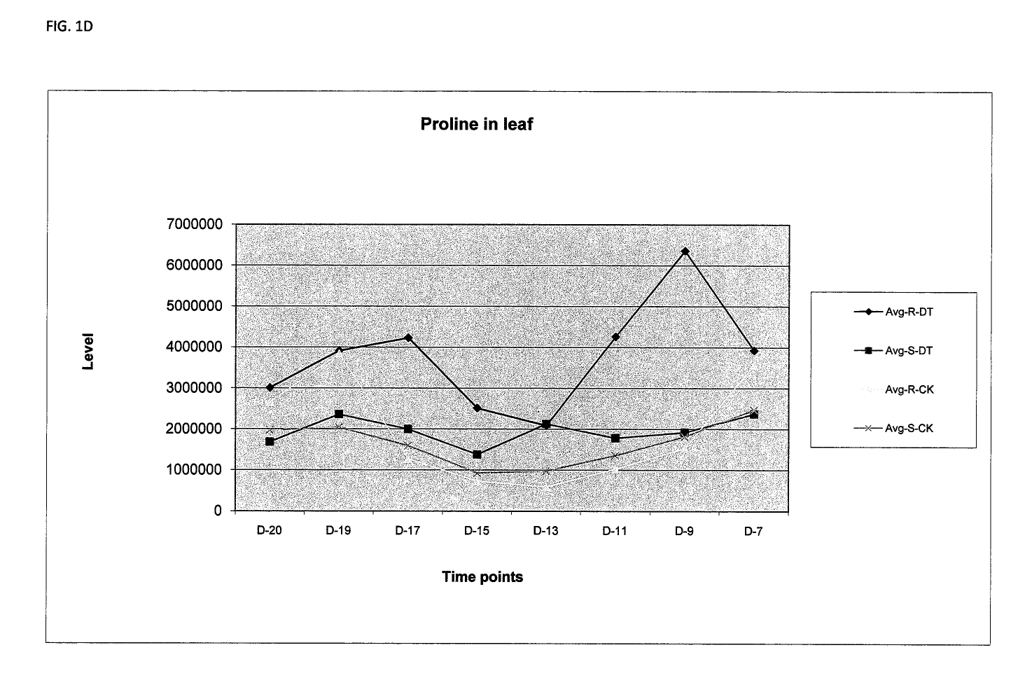

Drought treatment was started approximately 10 days before P50 (the time at which 50% of male flowers are already in bloom) and released at about 10 days after P50. Three out of 6 replicated plots were selected for sampling based on uniformity. Leaf samples were collected at midday to avoid interference with changes in expression due to diurnal rhythms. Tissues were collected at pre-flowering stages beginning with the initiation of stress and every 1-2 days thereafter. Tissues from 4 plants were pooled as one biological replicate sample for metabolite profiling. Phenotypic data on ASI (Anthesis-Silking Interval), barrenness, leaf rolling, leaf temperature, and grain yield were also recorded. Under drought conditions, levels of asparagine, glutamine, allantoin, and proline were increased. See FIGS. 1A-1D. It is known that allantoin is degraded to ureidolglycolate in most plant species. During this degradation process, ammonia is produced as a byproduct through multiple reaction steps and accumulated in plant vegetative tissue. It is well known that free ammonia increases in plant tissues during times of drought stress (see for example Blum et. al. 1976. Crop Science. 16: pp 428-431). The data as described in Table 1 and in FIGS. 1A-1D show that rather than an increase in the conversion of allantoin to it degradative byproducts (i.e. allantoate, ureidoglycine or ureidoglycolate) there is a trend at least in some lines to accumulate higher amounts of allantoin in drought conditions. From this data, it would seem that the plant has a preference to suppress the allantoin degradation pathway by inhibiting one or more of the enzymes associated with this pathway (i.e. allantoinase, allantoate amidohydrolase or ureidoglycolate amidohydrolase) and accumulate significant amounts of allantoin in its tissues during drought conditions.

### Example 3

### Generation of Transgenic Plants Having Increased Drought Tolerance

Two RNAi cassettes may be constructed for plant expression. These cassettes can be designed to down-regulate the enzymes allantoate amidohydrolase and allantoinase in maize vegetative and reproductive tissues to increase the accumulation of allantoin and to decrease the amount of ammonia accumulation therefore resulting in increased drought tolerance and/or yield. These RNAi cassettes can comprise a operably linked drought inducible promoter such as RAB17 (for example as described in Busk et. al. (1997) The Plant Journal 11(6) 1285-1295) or as depicted in SEQ ID NO: 1. Other drought inducible promoters may be used such as for example those described in U.S. Pat. No. 7,314,757 B2 herein incorporated by reference. A drought inducible promoter is preferred in that the constitutive expression of RNAi cassettes suppressing the allantoin degradation pathway may result in negative agronomic effects (e.g. tissue death, dwarfed plants, low seed set, etc). RNAi fragments (466 bp of zea mays allantoate amidohydrolase, SEQ ID NO: 2 and 401 bp of zea mays allantoinase, SEQ ID NO: 3) from the 3′-ends of the coding regions of the respective genes can be synthesized for example by a commercial DNA synthesis lab such as Geneart (Geneart AG). During synthesis, attB1 and attB2 sites can be added to 5′ and 3′ ends of the 2 RNAi sequences, respectively. In addition an intron from the rice sucrose synthase-1 (RSs1) gene is synthesized on the corresponding strand relative to the RNAi sequence to act as the spacer in the hair-pin loop formed by pairing the corresponding RNAi fragments, thus providing a binding region for the enzyme dicer. All RNAi expression cassettes can be recombined into a donor construct using the BP Reaction from Gateway® Cloning Technology (Invitrogen Life Science).

An expression cassette comprising a asparagine synthetase operably linked to drought inducible promoter (e.g. RAB17) can be constructed to increase levels of asparagine in drought conditions. For example, the asparagine synthetase gene as depicted in SEQ ID NO: 4 can be operably linked to the RAB17 promoter as depicted in SEQ ID NO: 1. A drought inducible promoter is preferred in that constitutive expression of asparagines synthetase may result in negative agronomic effects.

Destination vector 17593 is a binary vector containing a phosphomannose isomerase (PMI) gene that allows selection of transgenic cells with mannose. The Gateway Entry RNAi cassettes (SEQ ID #s 2-3) and the asparagines synthetase gene (SEQ ID NO: 4) can be recombined into vector 15910 using the LR Reaction (Gateway® LR Clonase® Enzyme Mix, Invitrogen). The drought inducible promoter of choice (e.g. RAB17) may then be cloned and operably linked to drive transcription of the respective RNAi or gene cassette as described above. Binary vectors can be verified by restriction digest and sequencing and then carried forward to plant transformation.

Transformation of immature maize embryos is performed essentially as described in Negrotto et al., Plant Cell Reports 19:798-803 (2000). Various media constituents described therein can be substituted.

Agrobacterium strain LBA4404 (Invitrogen) containing the plant transformation plasmid is grown on YEP (yeast extract (5 g/L), peptone (10 g/L), NaCl (5 g/L), 15 g/l agar, pH 6.8) solid medium for 2 to 4 days at 28° C. Approximately 0.8×109 Agrobacteria are suspended in LS-inf media supplemented with 100 μM acetosyringone (As) (LSAs medium) (Negrotto et al., Plant Cell Rep 19:798-803 (2000)). Bacteria are pre-induced in this medium for 30-60 minutes.

Immature embryos from maize line, A188, or other suitable maize genotypes are excised from 8-12 day old ears into liquid LS-inf+100 μM As (LSAs). Embryos are vortexed for 5 seconds and rinsed once with fresh infection medium. Infection media is removed and Agrobacterium solution is then added and embryos are vortexed for 30 seconds and allowed to settle with the bacteria for 5 minutes. The embryos are then transferred scutellum side up to LSAs medium and cultured in the dark for two to three days. Subsequently, between 20 and 25 embryos per petri plate are transferred to LSDc medium supplemented with cefotaxime (250 mg/l) and silver nitrate (1.6 mg/l) (Negrotto et al., Plant Cell Rep 19:798-803 (2000)) and cultured in the dark for 28° C. for 10 days.

Immature embryos producing embryogenic callus are transferred to LSD1M0.5S medium (LSDc with 0.5 mg/l 2, 4-D instead of Dicamba, 10 g/1 mannose, 5 g/l sucrose and no silver nitrate). The cultures are selected on this medium for 6 weeks with a subculture step at 3 weeks. Surviving calli are transferred either to LSD1M0.5S medium to be bulked-up or to Reg1 medium (as described in Negrotto et al., Plant Cell Rep 19:798-803 (2000)). Following culturing in the light (16 hour light/8 hour dark regiment), green tissues are then transferred to Reg2 medium without growth regulators (as described in Negrotto et al., Plant Cell Rep 19:798-803 (2000)) and incubated for 1-2 weeks. Plantlets are transferred to Magenta GA-7 boxes (Magenta Corp, Chicago Ill.) containing Reg3 medium (as described in Negrotto et al. (2000)) and grown in the light. Plants that are PCR positive for PMI and iOsSH 1 i1-01 (loop) and negative for Spectinomycin can be transferred to soil and grown in the greenhouse.

### Example 4

### Analysis of Drought Tolerant Events

### Drought Screen

Transgenic maize lines containing the expression cassette sequences as described in Example 3 can be screened for tolerance to drought stress in the following manner:

Transgenic maize plants are subjected to well-watered conditions (control) and to drought-stressed conditions. Transgenic maize plants are screened at the T1 stage or later. Stress is imposed starting at 10 to 14 days after sowing (DAS) or 7 days after transplanting, and is continued through to silking. Pots are watered by an automated system fitted to timers to provide watering at 25 or 50% of field capacity during the entire period of drought-stress treatment. The intensity and duration of this stress will allow identification of the impact on vegetative growth as well as on the anthesis-silking interval.

A mixture of ⅓ turface (Profile Products LLC, IL, USA), ⅓ sand and ⅓ SB300 (Sun Gro Horticulture, WA, USA) can be used. The SB300 can be replaced with Fafard Fine-Germ (Conrad Fafard, Inc., MA, USA) and the proportion of sand in the mixture can be reduced. Thus, a final potting mixture can be ⅜ (37.5%) turface, ⅜ (37.5%) Fafard and ¼ (25%) sand.

Field Capacity Determination: The weight of the soil mixture (w1) to be used in one S200 pot (minus the pot weight) is measured. If all components of the soil mix are not dry, the soil is dried at 100° C. to constant weight before determining w1. The soil in the pot is watered to full saturation and all the gravitational water is allowed to drain out. The weight of the soil (w2) after all gravitational water has seeped out (minus the pot weight) is determined. Field capacity is the weight of the water remaining in the soil obtained as w2−w1. It can be written as a percentage of the oven-dry soil weight.

Stress Treatment: During the early part of plant growth (10 DAS to 21 DAS), the well-watered control has a daily watering of 75% field capacity and the drought-stress treatment has a daily watering of 25% field capacity, both as a single daily dose at or around 10 AM. As the plants grow bigger, by 21 DAS, it will become necessary to increase the daily watering of the well-watered control to full field capacity and the drought stress treatment to 50% field capacity.

Nutrient Solution: A Hoagland's solution at 1/16 dilution with tap water is used for irrigation. Fertilizer grade KNO3 is used. It is useful to add half a teaspoon of Osmocote (NPK 15:9:12) to the pot at the time of transplanting or after emergence (The Scotts Miracle-Gro Company, OH, USA).

Border plants: Place a row of border plants on bench-edges adjacent to the glass walls of the greenhouse or adjacent to other potential causes of microenvironment variability such as a cooler fan.

Automation: Watering can be done using PVC pipes with drilled holes to supply water to systematically positioned pots using a siphoning device. Irrigation scheduling can be done using timers.

Statistical analysis: Mean values for plant size, color and chlorophyll fluorescence recorded on transgenic events under different stress treatments can be monitored. Treatment means will be evaluated for differences using Analysis of Variance.

Replications: Eight to ten individual plants are used per treatment per event.

Data Collection: From these test plants, biomass, chlorophyll levels, leaf rolling and various other methods well known in the art may be used to select transgenic plants with increased drought tolerance when compared to a control group. Subsequent yield analysis can be done to determine whether plants that contain the expression cassettes described in Example 3 have an improvement in yield performance under water-limiting conditions, when compared to the control plants. Specifically, drought conditions can be imposed during the flowering and/or grain fill period for transgenics and the control plants. Reduction in yield can be measured for both. Plants containing the expression cassettes of Example 3 may have less yield loss relative to the control plants, for example, 25% less yield loss.

Although the foregoing invention has been described in some detail by way of illustration and example for purposes of clarity and understanding, it will be clear to those of skill in the art that certain changes and modifications may be practiced within the scope of the appended claims.

1. A method of producing a transgenic plant with improved drought tolerance as compared to a non-transformed wild-type plant, the method comprising;
a) inserting an expression cassette into a plant cell comprising a polynucleotide operably linked to a drought inducible regulatory element that ensures the transcription of said polynucleotide in plant tissues in drought conditions wherein the transcription of said polynucleotide decreases the activity of one or more of the endogenous plant enzymes from the group consisting of allantoinase, allantoate amidohydrolase, and ureidoglycolate amidohydrolase;
b) regenerating a transgenic plant from the plant cell of a); and
c) producing said transgenic plant with improved drought tolerance.

2. The method of claim 1, wherein the plant is selected from the group consisting of soybean, sugar beet, cotton, maize, wheat, sorghum and sugarcane.

3. The method of claim 2, wherein the plant is maize.

4. The method of claim 1, wherein the drought inducible regulatory element is induced by abscisic acid, elevated levels of ammonia, elevated levels of proline or a ligand.

5. The method of claim 4, wherein the drought inducible promoter is a RAB17 promoter.

6. The method of claim 1, wherein the polynucleotide is a small interfering RNA or double-stranded RNA.

7. The method of claim 1, wherein the transgenic plant with improved drought tolerance does not comprise any negative agronomic effects.

8. The method of claim 1, wherein the transgenic plant confers improved yield in either drought or non-drought conditions.

9. A plant produced by the method of claim 1.

10. A method of producing a transgenic plant with improved drought tolerance as compared to a non-transformed wild-type plant, the method comprising;
a) inserting an expression cassette into a plant cell comprising a polynucleotide operably linked to a drought inducible regulatory element that ensures the transcription of said polynucleotide in plant tissues in drought conditions wherein the transcription of said polynucleotide increases the activity of one or more of the endogenous plant enzymes from the group consisting of glutamine synthetase, glutamate dehydrogenase, aspartate aminotransferase, and asparagines synthetase;
b) regenerating a transgenic plant from the plant cell of a); and
c) producing said transgenic plant with improved drought tolerance.

11. The method of claim 10, wherein the plant is selected from the group consisting of soybean, sugar beet, cotton, maize, wheat, sorghum and sugarcane.

12. The method of claim 11, wherein the plant is maize.

13. The method of claim 10, wherein the drought inducible regulatory element is induced by abscisic acid, elevated levels of ammonia, elevated levels of proline or a ligand.

14. The method of claim 13, wherein the drought inducible promoter is a RAB17 promoter.

15. The method of claim 10, wherein the transgenic plant with improved drought tolerance does not comprise any negative agronomic effects.

16. The method of claim 10, wherein the transgenic plant confers improved yield in either drought or non-drought conditions.


SEQUENCE LISTING (5 sequences)

SEQ ID NO: 1 - DNA, 598 bp, Zea mays
  promoter (1)..(598)
cttaattcgcccttataaactataatatttcaaaactatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcgcagatcctaatcctttcgccgttctgcacttctgctgcctataaatggcggcatcgaccgtcacctgcttcaccaccggcgagccacatcgagaacacgatcgagcacacaagcacgaagacta

SEQ ID NO: 2 - DNA, 466 bp, Zea mays
  iDNA (1)..(466)
gaagcgctgctgtagctggtacactacctgaatcaattttacaagtatcagataagagtggcactacactacaagatgttctgaaaatgaattcttttgaggctacctctactgctattagtcaagccaggtacaacccggagtctgtggggagttatgttgaggtacacatggagcaaggccctgtcctggaagcccttcattatccacttggtgttgtaaaaggaattgcaggacagacacgattgaaggtaatagtagatggttcgcaagggcatgctggcacagttccaatgaaattgcgccgtgatccgatggttgcagctgcagagctcgttgtgactctggaacgcctctgtaaagagcccaacagtttgctgacctacgacgaggagtgcagctgcttcactgaggagtccctggccggcctggtgtgcaccgtcggcgagctgaacacgtggccaag

SEQ ID NO: 3 - DNA, 401 bp, Zea mays
  iDNA (1)..(401)
acctcaaactaatggaggaaggggacttcttaagggcatggggaggcatatcatcattgcagtttgtcctcccggtgacatggtcatatgggcgaaagtatggcattactctgaatcagctagcctcatggtggagcgaaaggccagccaaacttgcaggccaaaagaataagggagctattctaccaggataccatgctgacattgtagtatggaaacccgaaacagagtttcaacttaacaataaccatgctatatatcataaacatcagaatatttcagcgtatttaggaaagcaactttctggtaaggtcctgtcgacttttgtgagaggaaatcttgtgtttgctgaagacaagcatgcaaatgctgcttgtggtgttcctatcctcgctaagtaa

SEQ ID NO: 4 - DNA, 1767 bp, Zea mays
  CDS (1)..(1767)
atgtgtggcatcttagccgtgctcggttgctccgactggtctcaggcaaagagggctcgcatcctcgcctgctccagaaggttgaagcacaggggccccgactggtcgggcctctaccagcacgagggcaacttcctggcgcagcagcggctcgccgtcgtctccccgctgtccggcgaccagccgctgttcaacgaggaccgcaccgtcgtggtggtggccaatggagagatctacaaccacaagaacgtccggaagcagttcaccggcacacacaacttcagcacgggcagtgactgcgaggtcatcatccccctgtacgagaagtacggcgagaacttcgtggacatgctggacggggtgttcgcgttcgtgctctacgacacccgcgacaggacctacgtggcggcgcgcgacgccatcggcgtcaacccgctctacatcggctggggcagtgacggttccgtctggatcgcgtccgagatgaaggcgctgaacgaggactgcgtgcgcttcgagatcttcccgccgggccacctctactccagcgccggcggcgggttccggcggtggtacaccccgcactggttccaggagcaggtgccccggatgccgtaccagccgctcgtcctcagagaggccttcgagaaggcggtcatcaagaggctcatgactgacgtcccgttcggggtcctcctctccggcggcctcgactcctcgctcgtcgcctccgtcaccaagcgccacctcgtcgagaccgaggccgccgagaagttcggcaccgagctccactcctttgtcgtcggcctcgagggctctcctgacctgaaggccgcacgagaggtcgctgactaccttggaaccatccatcacgagttccacttcaccgtacaggacggcatcgacgcgatcgaggaggtgatctaccacgacgagacgtacgacgtgacgacgatccgggccagcacgcccatgttcctgatggctcgcaagatcaagtcgctgggcgtgaagatggtgctgtccggggagggctccgacgagctcctgggcggctacctctacttccacttcgcccccaacaaggaggagttccacagggagacctgccgcaaggtgaaggccctgcaccagtacgactgcctgcgcgccaacaaggccacgtcggcgtggggcctggaggtccgcgtgccgttcctcgacaaggagttcatcaacgtcgcgatgggcatggaccccgaatggaaaatgtacgacaagaacctgggccgcatcgagaagtgggtcatgaggaaggcgttcgacgacgacgagcacccttacctgcccaagcatattctctacaggcagaaagaacagttcagtgacggcgttggctacaactggatcgatggcctcaaatccttcactgaacagcaggtgacggatgagatgatgaacaacgccgcccagatgttcccctacaacacgcccgtcaacaaggaggcctactactaccggatgatattcgagaggctcttccctcaggactcggcgagggagacggtgccgtggggcccgagcatcgcctgcagcacgcccgcggccatcgagtgggtggagcagtggaaggcctccaacgacccctccggccgcttcatctcctcccacgactccgccgccaccgaccacaccggcggtaagccggcggtggccaacggcggcggccacggcgccgcgaacggcacggtcaacggcaaggacgtcgcagtcgcgatcgcggtctga

SEQ ID NO: 5 - protein, 588 aa, Zea mays
MCGILAVLGCSDWSQAKRARILACSRRLKHRGPDWSGLYQHEGNFLAQQRLAVVSPLSGDQPLFNEDRTVVVVANGEIYNHKNVRKQFTGTHNFSTGSDCEVIIPLYEKYGENFVDMLDGVFAFVLYDTRDRTYVAARDAIGVNPLYIGWGSDGSVWIASEMKALNEDCVRFEIFPPGHLYSSAGGGFRRWYTPHWFQEQVPRMPYQPLVLREAFEKAVIKRLMTDVPFGVLLSGGLDSSLVASVTKRHLVETEAAEKFGTELHSFVVGLEGSPDLKAAREVADYLGTIHHEFHFTVQDGIDAIEEVIYHDETYDVTTIRASTPMFLMARKIKSLGVKMVLSGEGSDELLGGYLYFHFAPNKEEFHRETCRKVKALHQYDCLRANKATSAWGLEVRVPFLDKEFINVAMGMDPEWKMYDKNLGRIEKWVMRKAFDDDEHPYLPKHILYRQKEQFSDGVGYNWIDGLKSFTEQQVTDEMMNNAAQMFPYNTPVNKEAYYYRMIFERLFPQDSARETVPWGPSIACSTPAAIEWVEQWKASNDPSGRFISSHDSAATDHTGGKPAVANGGGHGAANGTVNGKDVAVAIAV